(19) United States
(12) Patent Application Publication
(10) Pub. No.: US 2020/0129330 A1
(43) Pub. Date: Apr. 30, 2020

(54) Phacoemulsification Needle

(71) Applicant: Douglas John Mastel, Rapid City, SD (US)

(72) Inventor: Douglas John Mastel, Rapid City, SD (US)

(21) Appl. No.: 16/664,844

(22) Filed: Oct. 26, 2019

Related U.S. Application Data
(60) Provisional application No. 62/751,374, filed on Oct. 26, 2018.

(51) Int. Cl. A61F 9/007; A61M 3/02; A61B 17/34

(52) U.S. Cl. CPC A61F 9/00745; A61M 3/0283; A61B 2017/3454; A61M 2210/0612; A61B 17/3421

(57) ABSTRACT

A phacoemulsification needle can include a head with a lumen, a shaft with a lumen, and a hub with a lumen, wherein the lumen diameter increases from the head to the hub. In some embodiments, the head of the phacoemulsification needle can be tapered such that the proximal end of the head is wider than the distal end of the head. In some embodiments, the distal end of the head has a beveled tip. In some embodiments, the sides of the head can be tapered and hexagonal, square, or triangular in cross-section. In some embodiments, the phacoemulsification needle can decrease the amount of ultrasonic energy, aspiration rate, or vacuum needed during phacoemulsification of a cataract.

### CROSS-REFERENCE TO RELATED APPLICATIONS

This application claims priority benefit from U.S. Application Ser. No. 62/751,374 filed on Oct. 26, 2018 entitled, “Phacoemulsification Needle”. The '374 application is hereby incorporated by reference herein in its entirety.

### FIELD OF THE INVENTION

The present invention relates to a medical device and, in particular, to a needle that can be used during phacoemulsification for the treatment of cataracts.

The lens of the eye is a transparent, biconvex structure composed primarily of water and crystallin. Situated behind the iris and pupil, the lens focuses incoming light onto the retina. The transparency of the lens is due to and maintained by the regression of the hyaloid vasculature after birth, the absence of light-scattering organelles within the mature lens fibers, the water-soluble nature of crystallin, and the diffusion of nutrients from the aqueous humor.

As the lens ages, old lens fibers are not replaced but rather new lens fibers are laid down over the existing crystallin. Over time, these aged, degraded or denatured crystallin proteins gradually accumulate to form cloudy aggregates, known as cataracts, that reduce light transmission of the lens.

Cataracts are the most common cause of vision loss in people over the age of forty, and the most common cause of blindness worldwide. An estimated 75 million people around the world will lose their vision due to cataracts by 2020.

Surgical intervention is the most common treatment for cataracts, with a high success rate for vision restoration. During surgery, an ophthalmologist removes the cloudy, cataract-afflicted lens of a patient and, in most cases, replaces it with a synthetic intraocular lens.

Phacoemulsification is a cataract surgery that utilizes an ultrasound probe to emulsify the lens of a cataract patient. The emulsified lens is then aspirated from the eye.

A phacoemulsification probe is an ultrasonic handpiece with a titanium or steel needle. The needle is introduced through an incision into the capsular lens. Once inserted, the needle vibrates at ultrasonic frequency to facilitate emulsification and aspiration.

Phacoemulsification probes can deliver ultrasound energy in a longitudinal manner, moving the needle forward and back, and/or rotational manner (also known as torsional or Ozil), moving the needle about the primary axis of the probe. Phacoemulsification needles or tips can be straight, flared, or curved, and the exterior distal portion of the needle or tip is smooth, round, and circular in cross-section.

The smooth, circular exterior can be disadvantageous during surgery, particularly during torsional movement of the needle. During sculpting and/or chopping of the lens, the smooth exterior surface of the needle is unable to engage and/or grip lenticular material as it rotates. This existing needle design can negatively impact occlusion and/or followability during phacoemulsification and can necessitate increased ultrasonic energy and/or vacuum strength which can make surrounding tissue more susceptible to trauma during surgery and/or increase the occurrence of postoperative complications.

A needle design with features to engage lenticular tissue during sculpting, chopping, and occlusion would be advantageous to patient outcome by preventing, or at least reducing, the need to increase ultrasonic energy and/or vacuum strength during phacoemulsification.

### SUMMARY OF THE INVENTION

A needle can include:
(a) a head with a first lumen formed therein;
(b) a shaft with a second lumen formed therein, with the shaft being continuous with the head;
(c) a hub with a third lumen formed therein, with the hub being continuous with the shaft, and wherein the diameter of the lumens increases from the head to the hub.

In some embodiments, the head of the needle is tapered and decreases in diameter from the proximal end to the distal end.

In some embodiments, the distal end of the head includes a beveled tip. In at least some embodiments, the tip is beveled at a 20°.

The head can include a plurality of sides that create an exterior surface that is hexagonal, square, or triangular in cross-section.

The needle can be attached to an ultrasonic phacoemulsification handpiece and used to emulsify and aspirate a cataract from the eye of a patient. In these embodiments, because the diameter of the needle lumen increases from the tip to the hub, the needle is prevented from clogging during aspiration.

A phacoemulsification needle can include:
(a) a head with a first lumen formed therein, the head including:
   (i) a proximal end;
   (ii) a distal end; and
   (iii) a plurality of tapered sides;
(b) a shaft with a second lumen formed therein, with the shaft being continuous with the head;
(c) a hub with a third lumen formed therein, with the hub being continuous with the shaft, and wherein the diameter of the lumens increases from the head to the hub.

In some embodiments, the head decreases in diameter from the proximal end to the distal end.

In some embodiments, the distal end of the head includes a beveled tip.

In some embodiments, the plurality of tapered sides can comprise six, four, or three sides.

In some embodiments, the needle can be attached to an ultrasonic phacoemulsification handpiece and used to emulsify and aspirate a cataract from the eye of a patient.

In at least some embodiments, the phacoemulsification needle can decrease the amount of ultrasonic energy, aspiration rate, and/or vacuum strength needed during sculpting, chopping, and/or occlusion of the cataract.

### BRIEF DESCRIPTION OF THE DRAWINGS

FIG. 1A is a side view a first embodiment of a phacoemulsification needle.

FIG. 1B is a cross-sectional view of the phacoemulsification needle of FIG. 1A.

FIG. 2A is an enlarged side view of a head of a first embodiment of a phacoemulsification needle.

FIG. 2B is an enlarged side view of the head of FIG. 2A illustrating dimensions.

FIG. 3 is a front view of a first embodiment of a phacoemulsification needle.

FIG. 4A is a front perspective view of a first embodiment of a phacoemulsification needle.

FIG. 4B is a rear perspective view of a first embodiment of a phacoemulsification needle.

FIG. 5A is a side view a second embodiment of a phacoemulsification needle.

FIG. 5B is a cross-sectional view of the phacoemulsification needle of FIG. 5A.

FIG. 6A is an enlarged side view of a head of a second embodiment of a phacoemulsification needle.

FIG. 6B is an enlarged side view of the head of FIG. 6A illustrating dimensions.

FIG. 7 is a front view of a second embodiment of a phacoemulsification needle.

FIG. 8A is a front perspective view of a second embodiment of a phacoemulsification needle.

FIG. 8B is a rear perspective view of a second embodiment of a phacoemulsification needle.

FIG. 9A is a side view a third embodiment of a phacoemulsification needle.

FIG. 9B is a cross-sectional view of the phacoemulsification needle of FIG. 9A.

FIG. 10A is an enlarged side view of a head of a third embodiment of a phacoemulsification needle.

FIG. 10B is an enlarged side view of the head of FIG. 10A illustrating dimensions.

FIG. 11 is a front view of a third embodiment of a phacoemulsification needle.

FIG. 12A is a front perspective view of a third embodiment of a phacoemulsification needle.

FIG. 12B is a rear perspective view of a third embodiment of a phacoemulsification needle.

FIG. 13A is a side view a fourth embodiment of a phacoemulsification needle.

FIG. 13B is a cross-sectional view of the phacoemulsification needle of FIG. 13A.

FIG. 14A is an enlarged side view of a head of a fourth embodiment of a phacoemulsification needle.

FIG. 14B is an enlarged side view of the head of FIG. 14A illustrating dimensions.

FIG. 15 is a front view of a fourth embodiment of a phacoemulsification needle.

FIG. 16A is a front perspective view of a fourth embodiment of a phacoemulsification needle.

FIG. 16B is a rear perspective view of a fourth embodiment of a phacoemulsification needle.

FIG. 17A is an enlarged perspective view of an embodiment of an expanded phacoemulsification needle head with internal, circumferential grooves.

FIG. 17B is an enlarged, transparent side view of the expanded phacoemulsification needle head of FIG. 17A.

FIG. 18A is an enlarged perspective view of an embodiment of an expanded phacoemulsification needle head with external, circumferential grooves.

FIG. 18B is an enlarged side view of the expanded phacoemulsification needle head of FIG. 18A.

FIG. 19 is an enlarged perspective view of an embodiment of a phacoemulsification needle head with external, longitudinal recesses.

FIG. 20 is an enlarged perspective view of an embodiment of an expanded phacoemulsification needle head with external, longitudinal recesses.

FIG. 21A is an enlarged perspective view of an embodiment of an expanded phacoemulsification needle head with a chamfered distal tip and external, longitudinal recesses.

FIG. 21B is an enlarged side view of the expanded phacoemulsification needle head of FIG. 21A.

FIG. 21C is a front view of the expanded phacoemulsification needle head of FIG. 21A.

FIG. 22A is an enlarged perspective view of another embodiment of an expanded phacoemulsification needle head with external, longitudinal recesses.

FIG. 22B is an enlarged front view of the expanded phacoemulsification needle head of FIG. 22A.

FIG. 23A is an enlarged perspective view of another embodiment of an expanded phacoemulsification needle head with external, longitudinal grooves.

FIG. 23B is an enlarged front view of the expanded phacoemulsification needle head of FIG. 23A.

FIG. 24A is an enlarged perspective view of an embodiment of an expanded phacoemulsification needle head with internal, longitudinal grooves.

FIG. 24B is an enlarged front view of the expanded phacoemulsification needle head of FIG. 24A.

FIG. 25A is an enlarged perspective view of another embodiment of an expanded phacoemulsification needle head with internal, longitudinal grooves.

FIG. 25B is an enlarged front view of the expanded phacoemulsification needle head of FIG. 25A.

FIG. 26A is an enlarged perspective view of an embodiment of an expanded phacoemulsification needle head with a convex shape.

FIG. 26B is an enlarged front view of the expanded phacoemulsification head of FIG. 26A.

FIG. 27A is an enlarged perspective view of an embodiment of an expanded phacoemulsification needle head with a concave shape.

FIG. 27B is an enlarged front view of the expanded phacoemulsification head of FIG. 27A.

FIG. 28A is an enlarged perspective view of an embodiment of an expanded phacoemulsification needle head with an internal chamfered edge.

FIG. 28B is an enlarged front view of the expanded phacoemulsification head of FIG. 28A.

FIG. 29 is a perspective view of a phacoemulsification needle illustrating the longitudinal and rotational motion of the tip during phacoemulsification.

FIG. 30 is an enlarged perspective view of an embodiment of an expanded phacoemulsification needle head with a set of exterior fins.

FIG. 31 is an enlarged side view of the expanded phacoemulsification needle head of FIG. 30.

FIG. 32 is an enlarged perspective view of an embodiment of an expanded phacoemulsification needle head with a set of exterior fins.

FIG. 33 is an enlarged side view of the expanded phacoemulsification needle head of FIG. 32.

FIG. 34 is an enlarged perspective view of an embodiment of an expanded phacoemulsification needle head with a set of exterior fins.

FIG. 35 is an enlarged side view of the expanded phacoemulsification needle head of FIG. 34.

FIG. 36 is an enlarged perspective view of an embodiment of an expanded phacoemulsification needle head with a set of exterior fins.

FIG. 37 is an enlarged side view of the expanded phacoemulsification needle head of FIG. 36.

FIG. 38 is an enlarged perspective view of an embodiment of an expanded phacoemulsification needle head with a set of exterior fins.

FIG. 39 is an enlarged side view of the expanded phacoemulsification needle head of FIG. 38.

### DETAILED DESCRIPTION OF ILLUSTRATIVE EMBODIMENT(S)

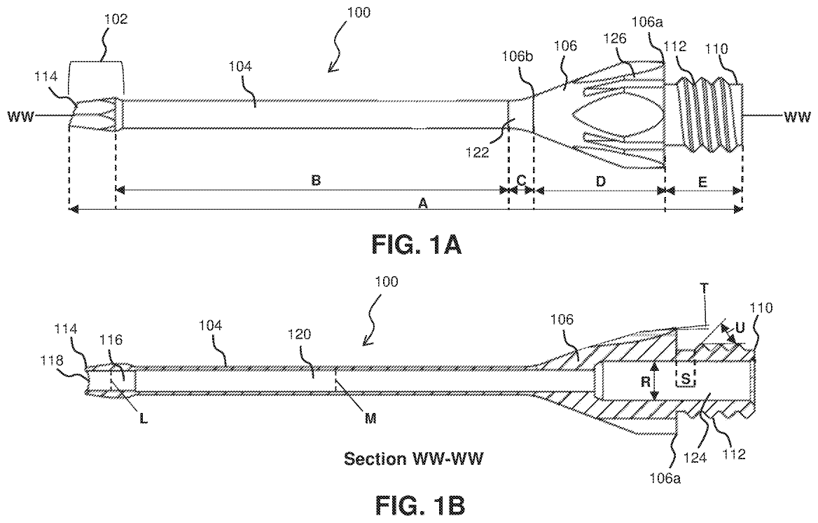

Turning first to FIGS. 1A and 1B, phacoemulsification needle 100 is shown. Phacoemulsification needle 100 can include head 102, shaft 104, and hub 106. In at least some embodiments, phacoemulsification needle 100 can be attached to an ultrasonic phacoemulsification handpiece at base 110 and used during cataract surgery to emulsify the lens of a patient and aspirate the emulsified lens from the eye. In some embodiments, base 110 can be attached to the ultrasonic phacoemulsification handpiece via threads 112. In some embodiments, threads 112 are 4-40 UNC-2A threads with a 0.38 mm, 45° chamfer (angle U in FIG. 1B). In at least some embodiments, head 102 can include at least one lumen 116 that is continuous with opening 118 formed at beveled tip 114 of head 102. In some embodiments, opening 118 functions as an aspiration port. In some embodiments, lumen 116 of head 102 can be continuous with at least one lumen 120 within shaft 104. In at least some embodiments, during phacoemulsification, aspiration port 118 and lumens 116 and 120 remove emulsified lens and loose debris from the eye using the vacuum pump function of the phacoemulsification machine.

In some embodiments, phacoemulsification needle 100 can be used with an infusion sleeve positioned around phacoemulsification needle 100 and/or an irrigation line positioned along shaft 104 and head 102. In at least some embodiments, during phacoemulsification, the infusion sleeve and/or irrigation line deliver balanced salt solution to the anterior chamber of the eye to supplement the aqueous humour removed during aspiration, therefore maintaining proper intraocular pressure.

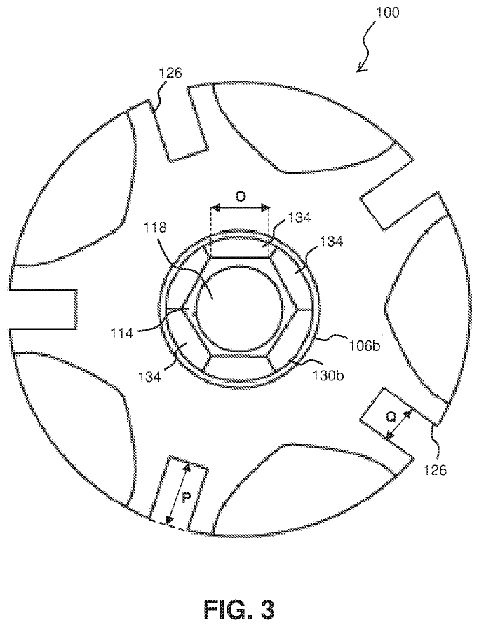

In some embodiments, hub 106 can include first end 106a and second end 106b. First end 106a can overhang base 110 of phacoemulsification needle 100. In some embodiments, the distance S from first end 106a to threads 112 can be approximately 0.69 mm. In at least some embodiments, second end 106b can be continuous with shaft 104. In some embodiments, the circumference of first end 106a is greater than the circumference of second end 106b. In some embodiments, hub 106 can include at least one groove 126. In some preferred embodiments, hub 106 can include five grooves 126. In some embodiments, such as when phacoemulsification needle 100 is used with an infusion sleeve, grooves 126 increase the flow of balanced salt solution to the eye. In some embodiments, such as that shown in FIG. 3, height P and width Q of groove(s) 126 can be approximately 0.48 mm and 0.30 mm, respectively. In some embodiments, grooves 126 can be equally spaced, 72°, about the circumference of hub 106. In some embodiments, Angle T of hub 106 (FIG. 1B) can be 15°. In some embodiments, angle T, can have a range between, and inclusive of, 5° and 25°. In some embodiments, grooves 126 decrease the mass of hub 106 and the overall mass of phacoemulsification needle 100. In certain embodiments, such as when phacoemulsification needle 100, or portions thereof, is made of metal, decreasing the mass of the needle via grooves 126 can reduce the amount of ultrasonic energy needed (intensity and/or duration) during phacoemulsification which can thereby prevent, or reduce, overheating of the needle.

In some preferred embodiments, total length A of phacoemulsification needle 100 can have a range between, and inclusive of, 21.62-22.12 mm. In some preferred embodiments, length A can be 21.87 mm.

In some embodiments, length B of shaft 104 can have a range between, and inclusive of, 12.75-13.25 mm. In some preferred embodiments, length B can be 13 mm.

In some embodiments, length C of proximal end 122 of shaft 104 can have a range between, and inclusive of, 0.87-1.07 mm. In some preferred embodiments, length C can be 0.97 mm.

In some embodiments, length D of hub 106 can have a range between, and inclusive of, 3.33-3.53 mm. In some preferred embodiments, length D can be 3.43 mm.

In some embodiments, length E of base 110 can have a range between, and inclusive of, 2.44-2.64 mm. In some preferred embodiments, length E can be 2.54 mm. In some embodiments, base 100 can terminate in chamfered edges.

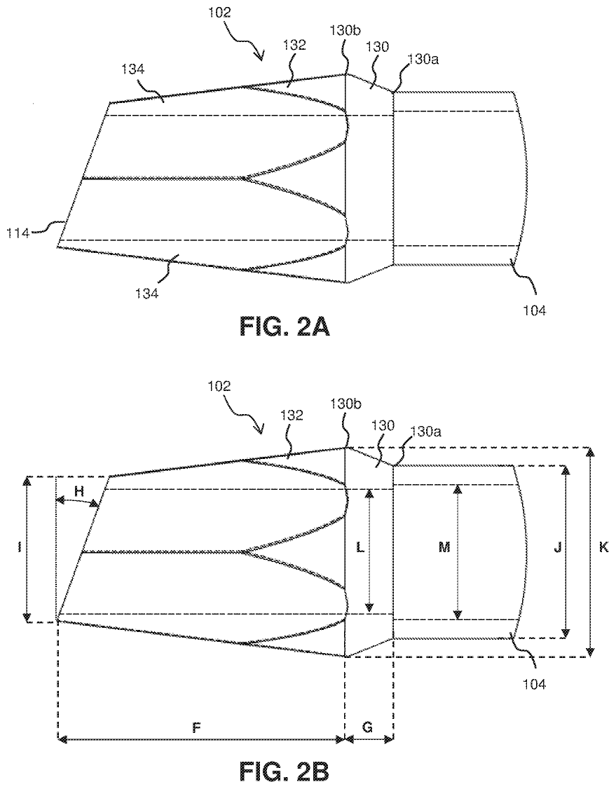

Turning to FIG. 2A, an enlarged view of head 102 is shown. In some embodiments, head 102 can include expanded portion 130 with first end 130a and second end 130b. In some embodiments, first end 130a of expanded portion 130 can be continuous with and have the same circumference as shaft 104. In some embodiments, second end 130b of expanded portion 130 can have a wider circumference than shaft 104. In some embodiments, head 102 can include tapered portion 132 which can have a proximal end (opposite beveled tip 114) that can be continuous with and/or have the same circumference as second end 130b of expanded portion 130. In some embodiments, tapered portion 132 can decrease in width from its proximal end to its distal end (beveled tip 114). In some embodiments, tapered portion 132 can include six sides 134 such that the exterior of tapered portion 132 is hexagonal in cross-section. Sides 134 of tapered portion 132 are shown in FIG. 3. In some embodiments, width O of side(s) 134 can have a range between, and inclusive of, 0.435-0.485 mm. In some preferred embodiments, width O can be 0.46 mm.

FIG. 2B illustrates dimensions of head 102 and shaft 104 for some embodiments of phacoemulsification needle 100.

In some embodiments, length F of tapered portion 132 can be 1.52 mm. In some embodiments, length G of expanded portion 130 can have a range between, and inclusive of, 0.385-0.435 mm. In some preferred embodiments, length G can be 0.41 mm.

In some embodiments, beveled tip 114 can be beveled such that angle H can have a range between, and inclusive of, 10° and 30°. In some embodiments, beveled tip 114 can be beveled such that angle H is 20°. In some embodiments, width I of beveled tip 114 of tapered portion 132 can have a range between, and inclusive of, 0.785-0.835 mm. In some preferred embodiments, width I can be 0.81 mm.

In some embodiments, width J of first end 130a of expanded portion 130 and shaft 104 can be 0.89 mm. In some embodiments, width K of second end 130b of expanded portion 130 and the proximal end of tapered portion 132 can have a range between, and inclusive of, 1.065-1.115 mm. In some preferred embodiments, width K can be 1.09 mm.

In some embodiments, the diameter of the lumens of phacoemulsification needle 100 increases from head 102 to shaft 104 to hub 106. In some preferred embodiments, diameter L of lumen 116 can be 0.024 0.61 mm, diameter M of lumen 120 can be 0.74 mm, and diameter R of lumen 124 can be 1.52 mm. In some embodiments, increasing the diameter of the lumens from the distal to the proximal end of phacoemulsification needle 100 can allow emulsified lens and debris to be removed from the eye during aspiration and moved the length of the needle with less resistance.

In some embodiments, the increasing lumen-diameter design from the distal end to the proximal end of phacoemulsification needle 100 can minimize, or at least reduce, clogging the lumens of phacoemulsification needle 100 due to aspiration during phacoemulsification. Specifically, by configuring aspiration port 118 and lumen 116 to be smaller in diameter than lumen 120 and/or lumen 124, the amount and/or size of lenticular material aspirated during phacoemulsification is limited or restricted at the distal end of the needle, tip 102, such that the proximal portions of the needle, shaft 104 and hub 106, do not receive an amount of lenticular material and/or lenticular material greater in size than can be handled by the length and diameter of lumen 120 and lumen 124. In at least some embodiments, the improved conductance of lenticular material during aspiration with phacoemulsification needle 100 overcomes the complications of existing phacoemulsification needle designs, including flare tip designs, in which the aspiration port and lumen at the distal end of the needle are greater in diameter than the proximal portions of the needle, the lumen of the shaft and/or the lumen of the hub, thereby making the needle more susceptible to clogging during aspiration.

In some embodiments, phacoemulsification needle 100 can prevent, or at least reduce, the need to increase the ultrasound energy and/or vacuum strength of a phacoemulsification machine for the purpose of dislodging a clog. This can prevent, or at least reduce, undesirable collateral damage to surrounding tissue including the iris and lens capsule.

During phacoemulsification, once phacoemulsification needle 100 is inserted through the incision made in the capsular lens, beveled tip 114 can contour the convex lens to deliver ultrasound energy and facilitate sculpting and/or chopping.

In at least some embodiments, sculpting refers to a phacoemulsification technique (also known as divide-and-conquer) in which an ophthalmologist divides the lens of a patient using low aspiration rate and vacuum settings with phaco (ultrasound) energy being modulated as needed based on lens density. Sculpting follows the convex contour of the lens with an initial superficial groove at the near periphery that deepens at the thicker lens center and returns superficially at the thinner far periphery (a first groove). The ophthalmologist repeats the sculpting technique to create an intersecting second groove perpendicular to the first groove to form a cross pattern and effectively divide the lens into quadrants. Aspiration of each quadrant can then be performed with minimal further delivery of ultrasound energy.

In at least some embodiments, chopping refers to a phacoemulsification technique in which an ophthalmologist applies higher aspiration rate and vacuum settings, relative to those used during sculpting, to fragment or chop lenticular material into smaller pieces prior to aspiration.

In some embodiments, beveled tip 114 can assist in lens grooving and cracking during phacoemulsification. In some preferred embodiments, beveled tip 114 can improve grooving and cracking along branched lens sutures such as Y sutures.

In some embodiments, the hexagonal nature of tapered portion 132 can allow sides 134 to interact with, engage, an/or grip target lenticular material during the delivery of ultrasound energy, particularly during rotation of beveled tip 114 about the longitudinal axis of phacoemulsification needle 100 (Ozil rotation), and promote occlusion of the material in aspiration port 118. Therefore, head 102 of phacoemulsification needle 100 can improve occlusion and followability during phacoemulsification. In some embodiments, the hexagonal exterior of tapered portion 132 overcomes the limitations of existing phacoemulsification needles which have distal ends that have circular exteriors and are, therefore, unable to engage and/or grip lenticular material during rotation of the distal ends about the longitudinal axis of the needles. In these existing needle designs, if a fragmented piece of lens has sharp edges, spinning it about the exterior of the distal end can cause trauma to surrounding tissue.

In at least some embodiments, occlusion refers to a phacoemulsification technique in which the ophthalmologist applies aspiration to the phacoemulsification needle to allow a portion of the cataract-afflicted lens of a patient to be brought to the tip of the needle such that the tip (aspiration port) becomes occluded with the lenticular material. Occlusion is typically achieved with higher aspiration rate and vacuum settings (400-600 mm-Hg), relative to those used during sculpting. Once lenticular material is captured by the tip of the needle, vacuum can be applied and increase over time (Adjustable Rise Time) to facilitate occlusion and followability.

In at least some embodiments, followability refers to bringing a fragment of lenticular material into the tip (aspiration port) of the phacoemulsification needle to cause occlusion and maintaining the occlusion as the lenticular material is aspirated out of the eye of the patient. Followability is a function of aspiration rate and vacuum settings as well as the geometry of the phacoemulsification needle.

In some embodiments, tapered portion 132 can assist in and improve lens grooving and cracking during phacoemulsification. In some preferred embodiments, tapered portion 132 can improve grooving and cracking along branched lens sutures such as Y sutures.

In some of embodiments, sides 134 of tapered portion 132 and/or beveled tip 114 can prevent, or at least reduce, undesirable collateral damage to surrounding tissue including the iris and lens capsule during phacoemulsification.

In some embodiments, sides 134 can also function as an infusion flow divertor by augmenting the path of balanced salt solution (BSS) from the infusion sleeve and/or irrigation line such that less BSS is distributed to opening 118. In some embodiments, excessive BSS can disrupt the ability of beveled tip 114 to aspirate lenticular material. The ability to divert infusion flow can improve occlusion and followability.

In some embodiments, phacoemulsification needle 100 can decrease the amount of ultrasound energy (movement), infusion or irrigation flow rate, aspiration rate, and/or vacuum needed during sculpting, chopping, and/or occlusion. This can prevent, or at least reduce the chance of, corneal incision contracture (corneal wound burn) resulting from ultrasound energy.

Phacoemulsification needle 100 can be compatible with various phacoemulsification techniques including, but not limited to, grooving, sculpting, cracking, divide-and-conquer, stop-and-chop, occlusion, and/or carousel.

In some embodiments, phacoemulsification needle 100 can prevent, or reduce, the occurrence of iris tears, capsular tears, and/or capsular rupture and vitreous loss during phacoemulsification.

In some embodiments, phacoemulsification needle 100 can minimize the occurrence of postoperative vitreoretinal complications such as retinal detachment, cystoid macular edema, endophthalmitis, and/or endothelial cell loss.

In at least some embodiments, during phacoemulsification, as beveled tip 114 penetrates into the lens nucleus, occlusion is initiated. As head 102 advances into lenticular tissue, the expanded (the proximal end of the head is larger in diameter than the shaft of the needle) and/or tapered nature of the head creates a wedge into the tissue and applies force to the material in front of advancing beveled tip 114 to initiate and propagate cracking of the lens.

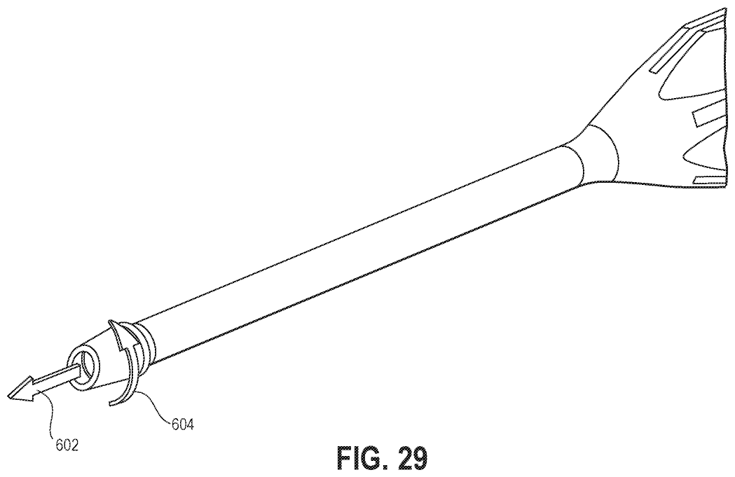

In some embodiments, the wedge effect of head 102 improves the efficacy of in longitudinal motion and/or rotational (Ozil) motion on lenticular tissue. FIG. 29 illustrates longitudinal motion (arrow 602) and Ozil motion (arrow 604) of phacoemulsification needle.

In some embodiments, the wedge effect of phacoemulsification needle 100 during occlusion improves the efficacy of chopping with a secondary instrument (such as a chopper).

In some embodiments, the wedge effect of phacoemulsification needle 100 can prevent, or reduce, the occurrence of coring. In some embodiments, phacoemulsification needle 100 can prevent, or reduce, the occurrence of post-occlusion surge of peripheral tissue such as the iris or capsule.

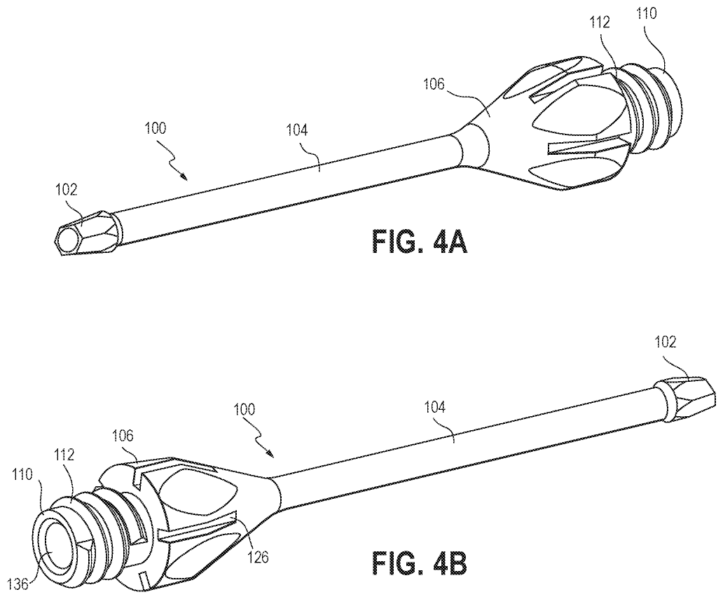

FIGS. 4A and 4B are perspective views of phacoemulsification needle 100. Opening 136 of lumen 124 can be seen in FIG. 4B.

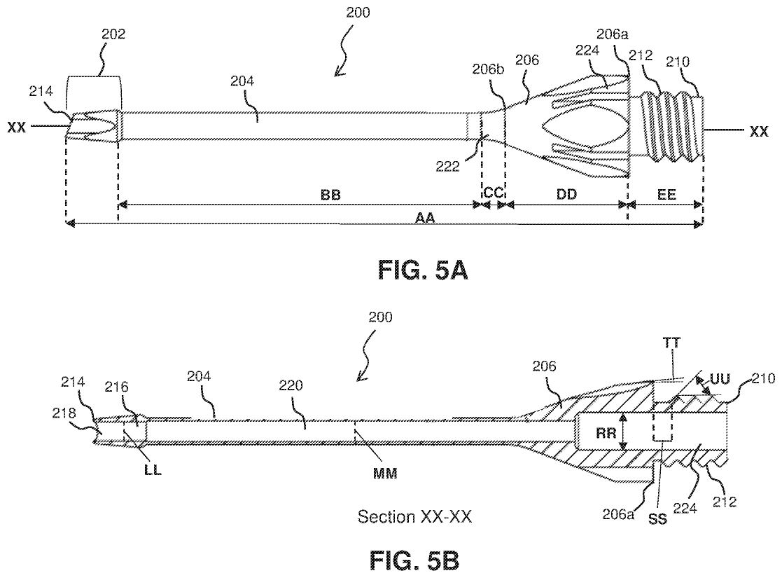

Another embodiment of a phacoemulsification needle is shown in FIGS. 5A and 5B. In some embodiments, phacoemulsification needle 200 can include head 202, shaft 204, and hub 206. In some embodiments, phacoemulsification needle 200 can be attached to an ultrasonic phacoemulsification handpiece at base 210 and used during cataract surgery to emulsify the lens of a patient and aspirate the emulsified lens from the eye. In some embodiments, base 210 can be attached to the ultrasonic phacoemulsification handpiece via threads 212. In some embodiments, threads 212 are 4-40 UNC-2A threads with a 0.38 mm, 45° chamfer (angle UU in FIG. 5B). In some embodiments, head 202 can include at least one lumen 216 that is continuous with opening 218 formed at beveled tip 214 of head 202. In some embodiments, opening 218 functions as an aspiration port. In some embodiments, lumen 216 of head 202 can be continuous with at least one lumen 220 within shaft 204. In some embodiments, during phacoemulsification, aspiration port 218 and lumens 216 and 220 remove emulsified lens and loose debris from the eye using the vacuum pump function of the phacoemulsification machine.

In some embodiments, phacoemulsification needle 200 can be used with an infusion sleeve positioned around phacoemulsification needle 200 or an irrigation line positioned along shaft 204 and head 202. In some embodiments, during phacoemulsification, the infusion sleeve or irrigation line deliver balanced salt solution to the anterior chamber of the eye to supplement the aqueous humour removed during aspiration, therefore maintaining proper intraocular pressure.

In some embodiments, hub 206 can include first end 206a and second end 206b. In some embodiments, first end 206a can overhang base 210 of phacoemulsification needle 200. In some embodiments, the distance SS from first end 206a to threads 212 can be approximately 0.69 mm. In some embodiments, second end 206b can be continuous with shaft 204. In some embodiments, the circumference of first end 206a can be greater than the circumference of second end 206b. In some embodiments, hub 206 can include at least one groove 226. In some preferred embodiments, hub 206 can include five grooves 226. In some embodiments, such as when phacoemulsification needle 200 is used with an infusion sleeve, grooves 226 increase the flow of balanced salt solution to the eye.

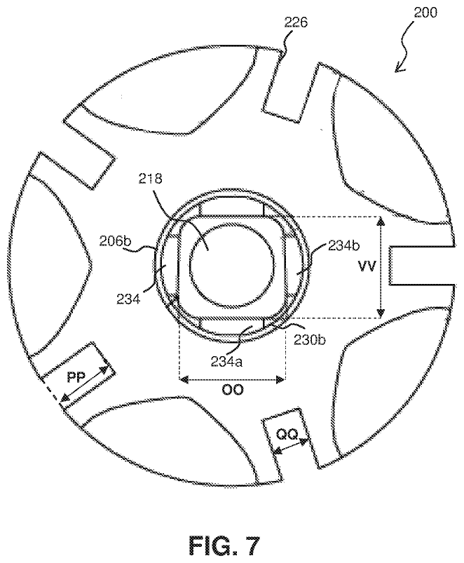

As shown in FIG. 7, height PP and width QQ of groove(s) 226 can be approximately 0.48 mm and 0.30 mm, respectively. In some embodiments, grooves 226 can be equally spaced, 72°, about the circumference of hub 206. In some embodiments, angle TT of hub 206 (FIG. 5B) can be 15°. In some embodiments, angle TT can have a range between, and inclusive of, 5° and 25°. In some embodiments, grooves 226 decrease the mass of hub 206 and the overall mass of phacoemulsification needle 200. In certain embodiments, such as when phacoemulsification needle 200, or portions thereof, is made of metal, decreasing the mass of the needle via grooves 226 can reduce the amount of ultrasonic energy needed (intensity and/or duration) during phacoemulsification which can thereby prevent, or reduce, overheating of the needle.

In some preferred embodiments, total length AA of phacoemulsification needle 200 can have a range between, and inclusive of, 21.62-22.12 mm. In some preferred embodiments, length AA can 21.87 mm.

In some embodiments, length BB of shaft 204 can have a range between, and inclusive of, 12.75-13.25 mm. In some preferred embodiments, length BB can be 13 mm.

In some embodiments, length CC of proximal end 222 of shaft 204 can have a range between, and inclusive of, 0.87-1.07 mm. In some preferred embodiments, length CC can be 0.97 mm.

In some embodiments, length DD of hub 206 can have a range between, and inclusive of, 3.33-3.53 mm. In some preferred embodiments, length DD can 3.43 mm.

In some embodiments, length EE of base 210 can have a range between, and inclusive of, 2.44-2.64 mm. In some preferred embodiments, length EE can be 2.54 mm. In some embodiments, base 200 can terminate in chamfered edges.

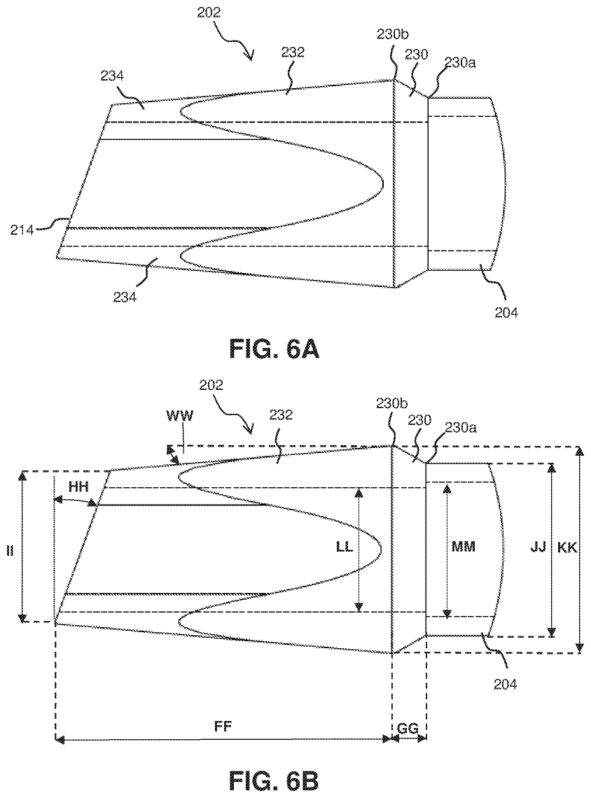

Turning to FIG. 6A, an enlarged view of head 202 is shown. In some embodiments, head 202 can include expanded portion 230 with first end 230a and second end 230b. In some embodiments, first end 230a of expanded portion 230 can be continuous with and have the same circumference as shaft 204. In some embodiments, second end 230b of expanded portion 230 can have a wider circumference than shaft 204. In some embodiments, head 202 can include tapered portion 232 which can have a proximal end (opposite beveled tip 214) that is continuous with and has the same circumference as second end 230b of expanded portion 230. In some embodiments, tapered portion 232 can decrease in width from its proximal end to its distal end (beveled tip 214). In some embodiments, tapered portion 232 can include four sides 234 such that the exterior of tapered portion 232 is square in cross-section. Sides 234 of tapered portion 232 are shown in FIG. 7.

In some embodiments, width OO of side 234a can have a range between, and inclusive of, 0.815-0.865 mm. In some embodiments, width VV of side 234b can have a range between, and inclusive of, 0.785-0.835 mm. In some preferred embodiments, width OO can be 0.84 mm and width VV can be 0.81 mm. In some embodiments, the radius of the rounded corners of sides 234 can be 0.15 mm.

FIG. 6B illustrates dimensions of head 202 and shaft 204 for some embodiments of phacoemulsification needle 200.

In some embodiments, length FF of tapered portion 232 can be 1.75 mm. In some embodiments, length GG of expanded portion 230 can have a range between, and inclusive of, 0.155-0.205 mm. In some preferred embodiments, length GG can be 0.18 mm.

In some embodiments, beveled tip 214 can be beveled such that angle HH is 20°. In some embodiments, angle HH can have a range between, and inclusive of, 10° and 30°. In some embodiments, width II of beveled tip 214 can have a range between, and inclusive of, 0.785-0.835 mm. In some preferred embodiments, width II can 0.81 mm.

In some embodiments, width JJ of first end 230a of expanded portion 230 and shaft 204 can be 0.89 mm. In some embodiments, width KK of second end 230b of expanded portion 230 and the proximal end of tapered portion 232 can have a range between, and inclusive of, 1.065-1.115 mm. In some preferred embodiments, width KK can 1.09 mm. In some embodiments, angle WW of tapered portion 232 can be 5°. In some embodiments, angle WW can have a range between, and inclusive of, 1° and 10°.

In some embodiments, the diameter of the lumens of phacoemulsification needle 200 can increase from head 202 to shaft 204 to hub 206. In some preferred embodiments, diameter LL of lumen 216 can be 0.66 mm, diameter MM of lumen 220 can be 0.74 mm, and diameter RR of lumen 224 can be 1.52 mm. In some embodiments, increasing the diameter of the lumens from the distal to the proximal end of phacoemulsification needle 200 can allow emulsified lens and debris to be removed from the eye during aspiration and moved the length of the needle with less resistance.

In some embodiments, the increasing lumen-diameter design from the distal end to the proximal end of phacoemulsification needle 200 can minimize, or reduce, clogging the lumens of phacoemulsification needle 200 due to aspiration during phacoemulsification. Specifically, by configuring aspiration port 218 and lumen 216 to be smaller in diameter than lumen 220 and/or lumen 224, the amount and/or size of lenticular material aspirated during phacoemulsification is limited or restricted at the distal end of the needle, tip 202, such that the proximal portions of the needle, shaft 204 and hub 206, do not receive an amount of lenticular material and/or lenticular material greater in size than can be handled by the length and diameter of lumen 220 and lumen 224. The improved conductance of lenticular material during aspiration with phacoemulsification needle 200 overcomes the complications of existing phacoemulsification needle designs, including flare tip designs, in which the aspiration port and lumen at the distal end of the needle are greater in diameter than the proximal portions of the needle, the lumen of the shaft and/or the lumen of the hub, thereby making the needle more susceptible to clogging during aspiration.

In some embodiments, phacoemulsification needle 200 can prevent, or reduce, the need to increase the ultrasound energy and/or vacuum strength of a phacoemulsification machine for the purpose of dislodging a clog. In some embodiments, this can prevent, or at least reduce, undesirable collateral damage to surrounding tissue including the iris and lens capsule.

In some embodiments, during phacoemulsification, once phacoemulsification needle 200 is inserted through the incision made through the capsular lens, beveled tip 214 can contour the convex lens to deliver ultrasound energy and facilitate sculpting and/or chopping.

In some embodiments, beveled tip 214 can assist in lens grooving and cracking during phacoemulsification. In some preferred embodiments, beveled tip 214 can improve grooving and cracking along branched lens sutures such as Y sutures.

In some embodiments, the square nature of tapered portion 232 can allow sides 234 to interact with, engage, and/or grip target lenticular material during the delivery of ultrasound energy, particularly during rotation of beveled tip 214 about the longitudinal axis of phacoemulsification needle 200 (Ozil rotation), and/or promote occlusion of the material in aspiration port 218. Therefore, head 202 of phacoemulsification needle 200 can improve occlusion and followability during phacoemulsification. In some embodiments, the square exterior of tapered portion 232 overcomes the limitations of existing phacoemulsification needles which have distal ends that have circular exteriors and are, therefore, unable to engage and/or grip lenticular material during rotation of the distal ends about the longitudinal axis of the needles.

In some embodiments, tapered portion 232 can assist in and improve lens grooving and cracking during phacoemulsification. In some preferred embodiments, tapered portion 232 can improve grooving and cracking along branched lens sutures such as Y sutures.

In some of embodiments, sides 234 and/or beveled tip 214 can prevent, or at least reduce, undesirable collateral damage to surrounding tissue including the iris and lens capsule during phacoemulsification.

In some embodiments, sides 234 of tapered portion 232 can also function as an infusion flow divertor by augmenting the path of BSS from the infusion sleeve or irrigation line such that less BSS is distributed to opening 218. In some embodiments, excessive BSS can disrupt the ability of beveled tip 214 to aspirate lenticular material. The ability to divert infusion flow can improve occlusion and followability.

In some embodiments, phacoemulsification needle 200 can decrease the amount of ultrasound energy (movement), aspiration rate, and/or vacuum needed during sculpting, chopping, and/or occlusion. This can prevent, or at least reduce the chance of, corneal incision contracture (corneal wound burn) resulting from ultrasound energy.

In at least some embodiments, phacoemulsification needle 200 can be compatible with various phacoemulsification techniques including, but not limited to, grooving, sculpting, cracking, divide-and-conquer, stop-and-chop, occlusion, and/or carousel.

In some preferred applications, phacoemulsification needle 200 can be used during sculpting and/or divide-and-conquer.

In some embodiments, phacoemulsification needle 200 can prevent, or reduce, the occurrence of iris tears, capsular tears, and/or capsular rupture and vitreous loss during phacoemulsification.

In some embodiments, phacoemulsification needle 200 can minimize the occurrence of postoperative vitreoretinal complications such as retinal detachment, cystoid macular edema, endophthalmitis, and/or endothelial cell loss.

In at least some embodiments, during phacoemulsification, as beveled tip 214 penetrates into the lens nucleus, occlusion is initiated. As head 202 advances into lenticular tissue, the expanded (the proximal end of the head is larger in diameter than the shaft of the needle) and/or tapered nature of the head creates a wedge into the tissue and applies force to the material in front of advancing beveled tip 214 to initiate and propagate cracking of the lens.

In some embodiments, the wedge effect of head 202 improves the efficacy of longitudinal motion and/or rotational (Ozil) motion on lenticular tissue.

In some embodiments, the wedge effect of phacoemulsification needle 200 during occlusion improves the efficacy of chopping with a secondary instrument (such as a chopper).

In some embodiments, the wedge effect of phacoemulsification needle 200 can prevent, or reduce, the occurrence of coring. In some embodiments, phacoemulsification needle 200 can prevent, or reduce, the occurrence of post-occlusion surge of peripheral tissue such as the iris or capsule.

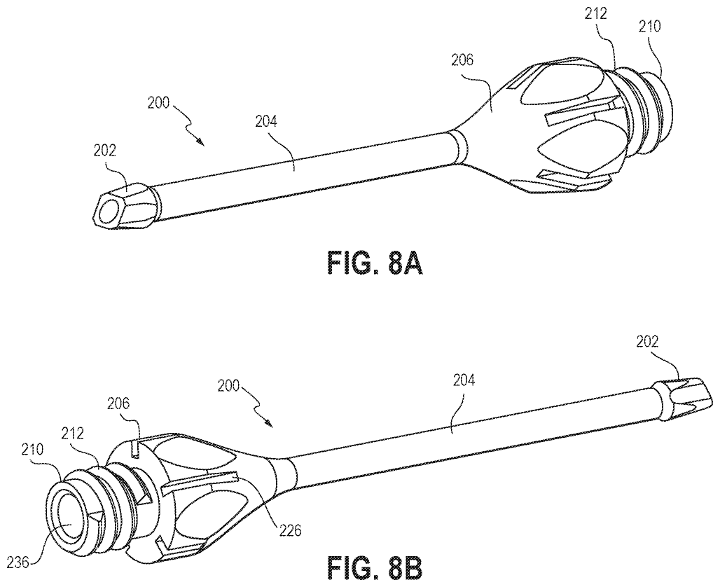

FIGS. 8A and 8B are perspective views of phacoemulsification needle 200. Opening 236 of lumen 224 can be seen in FIG. 8B.

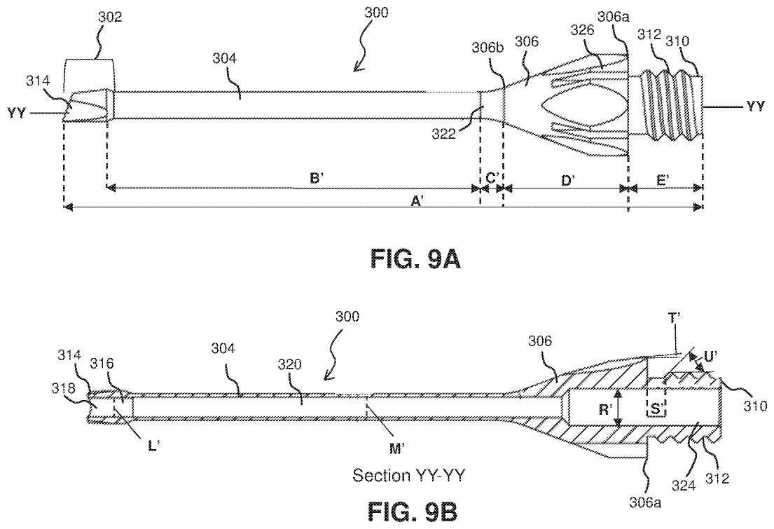

Another embodiment of a phacoemulsification needle is shown in FIGS. 9A and 9B. In some embodiments, phacoemulsification needle 300 can include head 302, shaft 304, and hub 306. In some embodiments, phacoemulsification needle 300 can be attached to an ultrasonic phacoemulsification handpiece at base 310 and used during cataract surgery to emulsify the lens of a patient and aspirate the emulsified lens from the eye. In some embodiments, base 310 can be attached to the ultrasonic phacoemulsification handpiece via threads 312. In some embodiments, threads 312 are 4-40 UNC-2A threads with a 0.38 mm, 45° chamfer (angle U′ in FIG. 9B). In some embodiments, head 302 can include at least one lumen 316 that is continuous with opening 318 formed at beveled tip 314 of head 302. In some embodiments, opening 318 functions as an aspiration port. In some embodiments, lumen 316 of head 302 can be continuous with at least one lumen 320 within shaft 304. In some embodiments, during phacoemulsification, aspiration port 318 and lumens 316 and 320 remove emulsified lens and loose debris from the eye using the vacuum pump function of the phacoemulsification machine.

In some embodiments, phacoemulsification needle 300 can be used with an infusion sleeve positioned around phacoemulsification needle 300 and/or an irrigation line positioned along shaft 304 and head 302. In some embodiments, during phacoemulsification, the infusion sleeve and/or irrigation line deliver balanced salt solution to the anterior chamber of the eye to supplement the aqueous humour removed during aspiration, therefore maintaining proper intraocular pressure.

In some embodiments, hub 306 can include first end 306a and second end 306b. In some embodiments, first end 306a can overhang base 310 of phacoemulsification needle 300. In some embodiments, the distance S′ from first end 306a to threads 312 can be approximately 0.69 mm. In some embodiments, second end 306b can be continuous with shaft 304. In some embodiments, the circumference of first end 306a can be greater than the circumference of second end 306b.

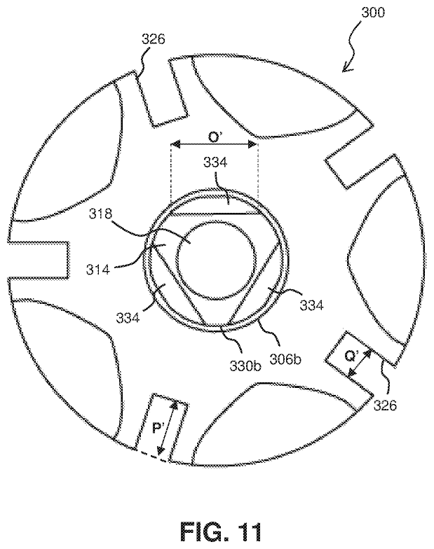

In some embodiments, hub 306 can include at least one groove 326. In some preferred embodiments, hub 306 can include five grooves 326. In some embodiments, such as when phacoemulsification needle 300 is used with an infusion sleeve, grooves 326 increase the flow of balanced salt solution to the eye. As shown in FIG. 11, height P′ and width Q′ of groove(s) 326 can be approximately 0.51 mm and 0.30 mm, respectively. In some embodiments, grooves 326 can be equally spaced, 72°, about the circumference of hub 306. Angle T′ of hub 306 (FIG. 9B) can be 15°. In some embodiments, angle T′ can have a range between, and inclusive of, 5° to 25°. Grooves 326 decrease the mass of hub 306 and the overall mass of phacoemulsification needle 300. In certain embodiments, such as when phacoemulsification needle 300, or portions thereof, is made of metal, decreasing the mass of the needle via grooves 126 can reduce the amount of ultrasonic energy needed (intensity and/or duration) during phacoemulsification which can thereby prevent, or reduce, overheating of the needle.

In some preferred embodiments, total length A′ of phacoemulsification needle 300 can have a range between, and inclusive of, 21.62-22.12 mm. In some preferred embodiments, length A′ can be 21.87 mm.

In some embodiments, length B′ of shaft 304 can have a range between, and inclusive of, 12.75-13.25 mm. In some preferred embodiments, length B′ can be 13.18 mm.

In some embodiments, length C′ of proximal end 322 of shaft 304 can have a range between, and inclusive of, 0.87-1.07 mm. In some preferred embodiments, length C′ can be 0.97 mm.

In some embodiments, length D′ of hub 306 can have a range between, and inclusive of, 3.33-3.53 mm. In some preferred embodiments, length D′ can be 3.43 mm.

In some embodiments, length E′ of base 310 can have a range between, and inclusive of, 2.44-2.64 mm. In some preferred embodiments, length E′ can be 2.54 mm. Base 300 can terminate in chamfered edges.

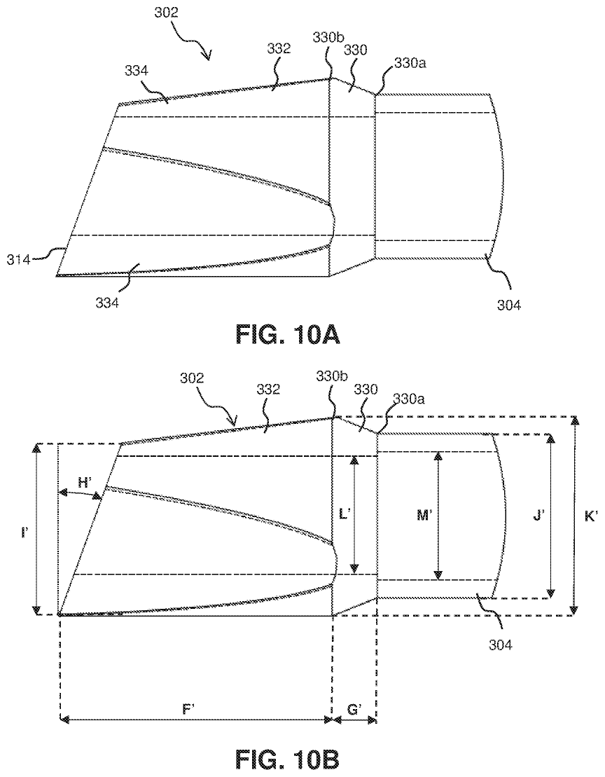

Turning to FIG. 10A, an enlarged view of head 302 is shown. In some embodiments, head 302 can include expanded portion 330 with first end 330a and second end 330b. In some embodiments, first end 330a of expanded portion 330 can be continuous with and has the same circumference as shaft 304. In some embodiments, second end 330b of expanded portion 330 can have a wider circumference than shaft 304. In some embodiments, head 302 can include tapered portion 332 which can have a proximal end (opposite beveled tip 314) that is continuous with and has the same circumference as second end 330b of expanded portion 330. In some embodiments, tapered portion 332 can decrease in width from its proximal end to its distal end (beveled tip 314). In some embodiments, tapered portion 332 can include three sides 334 such that the exterior of tapered portion 332 is triangular in cross-section. Sides 334 of tapered portion 332 are shown in FIG. 11. In some embodiments, width O′ of side(s) 334 can have a range between, and inclusive of, 0.735-0.785 mm. In some preferred embodiments, width O′ can be 0.76 mm.

FIG. 10B illustrates dimensions of head 302 and shaft 304 for some embodiments of phacoemulsification needle 300.

In some embodiments, length F′ of tapered portion 332 can be 1.52 mm. In some embodiments, length G′ of expanded portion 330 can have a range between, and inclusive of, 0.205-0.255 mm. In some preferred embodiments, length G′ can be 0.23 mm.

In some embodiments, beveled tip 314 can be beveled such that angle H′ is 20°. In some embodiments, angle H′ can have a range between, and inclusive of 10° and 30°. In some embodiments, width I′ of beveled tip 314 of tapered portion 332 can have a range between, and inclusive of, 0.785-0.835 mm. In some preferred embodiments, width I′ can be 0.81 mm.

In some embodiments, width J′ of first end 330a of expanded portion 330 and shaft 304 can be 0.89 mm. In some embodiments, width K′ of second end 330b of expanded portion 330 and the proximal end of tapered portion 332 can have a range between, and inclusive of, 0.995-1.045 mm. In some preferred embodiments, width K′ can be 1.02 mm.

In some embodiments, the diameter of the lumens of phacoemulsification needle 300 can increase from head 302 to shaft 304 to hub 306. In some preferred embodiments, diameter L′ of lumen 316 can be 0.66 mm, diameter M′ of lumen 320 can be 0.74 mm, and diameter R′ of lumen 324 can be 1.52 mm. In some embodiments, increasing the diameter of the lumens from the distal to the proximal end of phacoemulsification needle 300 can allow emulsified lens and debris to be removed from the eye during aspiration and moved the length of the needle with less resistance.

In some embodiments, the increasing lumen-diameter design from the distal end to the proximal end of phacoemulsification needle 300 can minimize, or reduce, clogging the lumens of phacoemulsification needle 300 due to aspiration during phacoemulsification. Specifically, by configuring aspiration port 318 and lumen 316 to be smaller in diameter than lumen 320 and/or lumen 324, the amount and/or size of lenticular material aspirated during phacoemulsification is limited or restricted at the distal end of the needle, tip 302, such that the proximal portions of the needle, shaft 304 and hub 306, do not receive a greater amount of lenticular material and/or lenticular material greater in size than can be handled by the length and diameter of lumen 320 and lumen 324. In some embodiments, the improved conductance of lenticular material during aspiration with phacoemulsification needle 300 overcomes the complications of existing phacoemulsification needle designs, including flare tip designs, in which the aspiration port and lumen at the distal end of the needle are greater in diameter than the proximal portions of the needle, the lumen of the shaft and/or the lumen of the hub, thereby making the needle more susceptible to clogging during aspiration.

In some embodiments, phacoemulsification needle 300 can prevent, or reduce, the need to increase the ultrasound energy and/or vacuum strength of a phacoemulsification machine for the purpose of dislodging a clog. In at least some embodiments, this can prevent, or at least reduce, undesirable collateral damage to surrounding tissue including the iris and lens capsule.

In some embodiments, during phacoemulsification, once phacoemulsification needle 300 is inserted through the incision made through the capsular lens, beveled tip 314 can contour the convex lens to deliver ultrasound energy and facilitate sculpting and/or chopping.

In some embodiments, beveled tip 314 can assist in lens grooving and cracking during phacoemulsification. In some preferred embodiments, beveled tip 314 can improve grooving and cracking along branched lens sutures such as Y sutures.

In some embodiments, the triangular nature of tapered portion 332 can allow sides 334 to interact with, engage and/or grip target lenticular material during the delivery of ultrasound energy, particularly during rotation of beveled tip 314 about the longitudinal axis of phacoemulsification needle 300 (Ozil rotation), and promote occlusion of the material in aspiration port 318. Therefore, head 302 of phacoemulsification needle 300 can improve occlusion and followability during phacoemulsification. In some embodiments, the triangular exterior of tapered portion 332 overcomes the limitations of existing phacoemulsification needles which have distal ends that have circular exteriors and are, therefore, unable to engage and/or grip lenticular material during rotation of the distal ends about the longitudinal axis of the needles.

In some embodiments, tapered portion 332 can assist in and improve lens grooving and cracking during phacoemulsification. In some preferred embodiments, tapered portion 332 can improve grooving and cracking along branched lens sutures such as Y sutures.

In some of embodiments, sides 334 and/or beveled tip 314 can prevent, or at least reduce, undesirable collateral damage to surrounding tissue including the iris and lens capsule during phacoemulsification.

Sides 334 of tapered portion 332 can also function as an infusion flow divertor by augmenting the path of balanced salt solution (BSS) from the infusion sleeve and/or irrigation line such that less BSS is distributed to opening 318. Excessive BSS can disrupt the ability of beveled tip 314 to aspirate lenticular material. The ability to divert infusion flow can improve occlusion and followability.

In some embodiments, phacoemulsification needle 300 can decrease the amount of ultrasound energy (movement), aspiration rate, and/or vacuum needed during sculpting, chopping, and/or occlusion. In some embodiments, this can prevent, or at least reduce the chance of, corneal incision contracture (corneal wound burn) resulting from ultrasound energy.

In some embodiments, phacoemulsification needle 300 can be compatible with various phacoemulsification techniques including, but not limited to, grooving, sculpting, cracking, divide-and-conquer, stop-and-chop, occlusion, and/or carousel.

In some preferred applications, phacoemulsification needle 300 can be used during cracking and chopping as the triangular nature of tapered portion 332 of head 302 can crack the embryonic plates of the three lens lobes joined by the Y suture.

In some embodiments, phacoemulsification needle 300 can prevent, or at least reduce the chance of, iris tears, capsular tears, and/or capsular rupture and vitreous loss during phacoemulsification.

In some embodiments, phacoemulsification needle 300 can minimize, or at least reduce, the occurrence of postoperative vitreoretinal complications such as retinal detachment, cystoid macular edema, endophthalmitis, and/or endothelial cell loss.

In at least some embodiments, during phacoemulsification, as beveled tip 314 penetrates into the lens nucleus, occlusion is initiated. As head 302 advances into lenticular tissue, the expanded (the proximal end of the head is larger in diameter than the shaft of the needle) and/or tapered nature of the head creates a wedge into the tissue and applies force to the material in front of advancing beveled tip 314 to initiate and propagate cracking of the lens.

In some embodiments, the wedge effect of head 302 improves the efficacy of in longitudinal motion and/or rotational (Ozil) motion on lenticular tissue.

In some embodiments, the wedge effect of phacoemulsification needle 300 during occlusion improves the efficacy of chopping with a secondary instrument (such as a chopper).

In some embodiments, the wedge effect of phacoemulsification needle 300 can prevent, or reduce, the occurrence of coring. In some embodiments, phacoemulsification needle 300 can prevent, or reduce, the occurrence of post-occlusion surge of peripheral tissue such as the iris and/or capsule.

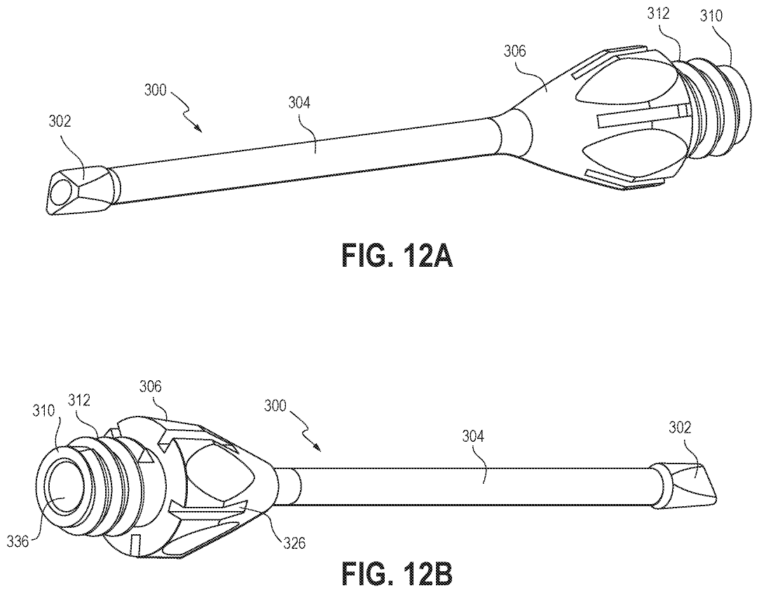

FIGS. 12A and 12B are perspective views of phacoemulsification needle 300. Opening 336 of lumen 324 can be seen in FIG. 12B.

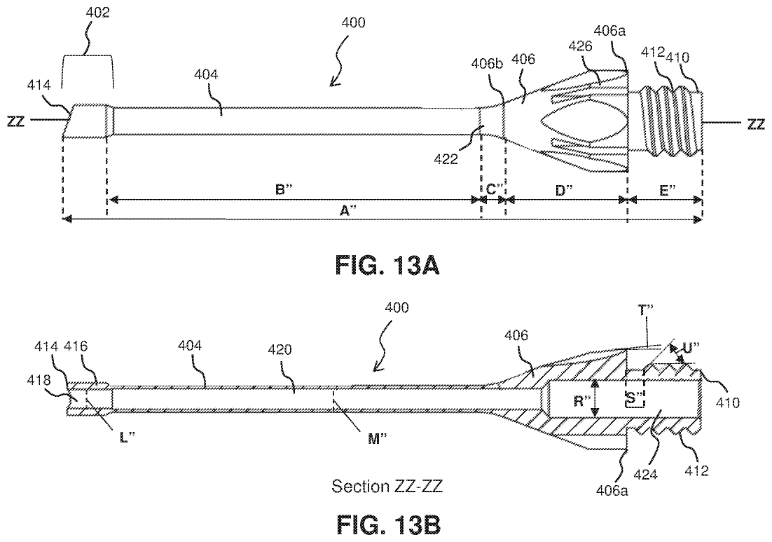

Another embodiment of a phacoemulsification needle is shown in FIGS. 13A and 913B. In some embodiments, phacoemulsification needle 400 can include head 402, shaft 404, and/or hub 406. In some embodiments, phacoemulsification needle 400 can be attached to an ultrasonic phacoemulsification handpiece at base 410 and used during cataract surgery to emulsify the lens of a patient and aspirate the emulsified lens from the eye. In some embodiments, base 410 can be attached to the ultrasonic phacoemulsification handpiece via threads 412. In some embodiments, threads 412 are 4-40 UNC-2A threads with a 0.38 mm, 45° chamfer (angle U″ in FIG. 13B). In some embodiments, head 402 can include at least one lumen 416 that is continuous with opening 418 formed at beveled tip 414 of head 402. In some embodiments, opening 418 functions as an aspiration port. In some embodiments, lumen 416 of head 402 can be continuous with at least one lumen 420 within shaft 404. In some embodiments, during phacoemulsification, aspiration port 418 and lumens 416 and 420 remove emulsified lens and loose debris from the eye using the vacuum pump function of the phacoemulsification machine.

In some embodiments, phacoemulsification needle 400 can be used with an infusion sleeve positioned around phacoemulsification needle 400 and/or an irrigation line positioned along shaft 404 and head 402. During phacoemulsification, the infusion sleeve and/or irrigation line deliver balanced salt solution to the anterior chamber of the eye to supplement the aqueous humour removed during aspiration, therefore maintaining proper intraocular pressure.

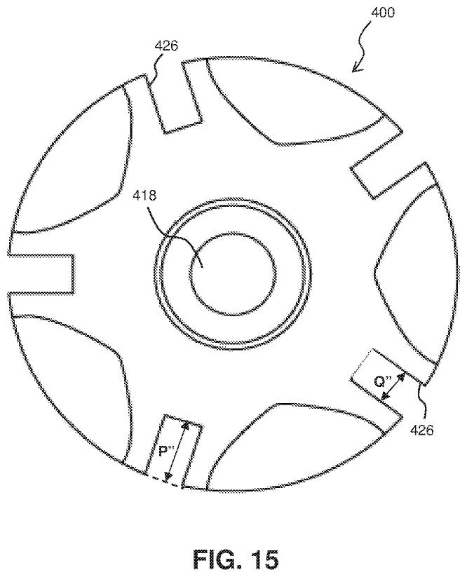

In some embodiments, hub 406 can include first end 406a and second end 406b. In some embodiments, first end 406a can overhang base 410 of phacoemulsification needle 400. In some embodiments, the distance S″ from first end 406a to threads 412 can be approximately 0.69 mm. In some embodiments, second end 406b can be continuous with shaft 404. In some embodiments, the circumference of first end 406a can be greater than the circumference of second end 406b. In some embodiments, hub 406 can include at least one groove 426. In some preferred embodiments, hub 406 can include five grooves 426. In some embodiments, such as when phacoemulsification needle 400 is used with an infusion sleeve, grooves 426 increase the flow of balanced salt solution to the eye. As shown in FIG. 15, height P″ and width Q″ of groove(s) 426 can be approximately 0.51 mm and 0.30 mm, respectively. In some embodiments, grooves 426 can be equally spaced, 72°, about the circumference of hub 406. In some embodiments, angle T″ of hub 406 (FIG. 13B) can be 15°. In some embodiments, angle T″ can have a range between, and inclusive of 5° and 25°. In some embodiments, grooves 426 decrease the mass of hub 406 and the overall mass of phacoemulsification needle 400. In certain embodiments, such as when phacoemulsification needle 400, or portions thereof, is made of metal, decreasing the mass of the needle via grooves 426 can reduce the amount of ultrasonic energy needed (intensity and/or duration) during phacoemulsification which can thereby prevent, or reduce, overheating of the needle.

In some preferred embodiments, total length A″ of phacoemulsification needle 400 have a range between, and inclusive of, 21.62-22.12 mm. In some preferred embodiments, length A″ can be 21.87 mm.

In some embodiments, length B″ of shaft 404 can have a range between, and inclusive of, 12.75-13.25 mm. In some preferred embodiments, length B″ can be 13.18 mm.

In some embodiments, length C″ of proximal end 422 of shaft 404 can have a range between, and inclusive of, 0.87-1.07 mm. In some preferred embodiments, length C″ can be 0.97 mm.

In some embodiments, length D″ of hub 406 can have a range between, and inclusive of, 3.33-3.53 mm. In some preferred embodiments, length D″ can be 3.43 mm.

In some embodiments, length E″ of base 410 can have a range between, and inclusive of, 2.44-2.64 mm. In some preferred embodiments, length E″ can be 2.54 mm. Base 400 can terminate in chamfered edges.

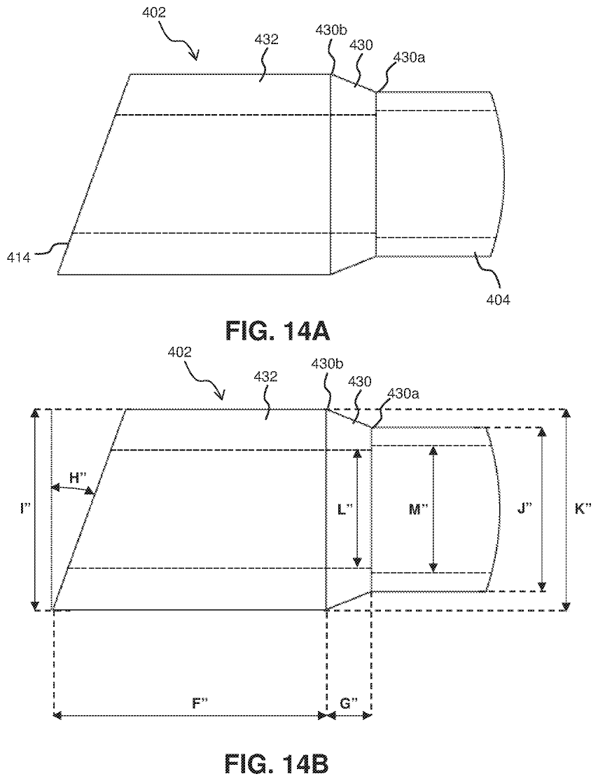

Turning to FIG. 14A, an enlarged view of head 402 is shown. In some embodiments, head 402 can include neck 430, first end 430a and second end 430b. In some embodiments, first end 430a of neck 430 can be continuous with and have the same circumference as shaft 404. In some embodiments, second end 430b of neck 430 can have a wider circumference than shaft 404. In some embodiments, head 402 can include portion 432 which can have a proximal end (opposite beveled tip 414) that is continuous with and has the same circumference as second end 430b of neck 430. In some embodiments, portion 432 can be tapered and decrease in width from its proximal end to its distal end (beveled tip 414). In some embodiments, portion 432 can be tapered and decrease in width from the proximal end to the distal end.

FIG. 14B illustrates dimensions of head 402 and shaft 404 for some embodiments of phacoemulsification needle 400.

In some embodiments, length F″ of portion 432 can be 1.52 mm. In some embodiments, length G″ of neck 430 can be 0.23 mm.

In some embodiments, beveled tip 414 can be beveled such that angle H″ is 20°. In some embodiments, width I″ of beveled tip 414 can have a range between, and inclusive of, 0.995-1.045 mm. In some preferred embodiments, width I″ can be 1.02 mm.

In some embodiments, width J″ of first end 430a of neck 430 and shaft 404 can be 0.89 mm. In some embodiments, width K″ of second end 430b of neck 430 can have a range between, and inclusive of, 0.995-1.045 mm. In some preferred embodiments, width K″ can be 1.02 mm.

In some embodiments, the diameter of the lumens of phacoemulsification needle 400 can increase from head 402 to shaft 404 to hub 406. In some preferred embodiments, diameter L″ of lumen 416 can be 0.66 mm, diameter M″ of lumen 420 can be 0.74 mm, and diameter R″ of lumen 424 can be 1.52 mm. In some embodiments, increasing the diameter of the lumens from the distal to the proximal end of phacoemulsification needle 400 can allow emulsified lens and debris to be removed from the eye during aspiration and moved the length of the needle with less resistance.

In some embodiments, the increasing lumen-diameter design from the distal end to the proximal end of phacoemulsification needle 400 can minimize, or at least reduce, clogging the lumens of phacoemulsification needle 400 due to aspiration during phacoemulsification. Specifically, by configuring aspiration port 418 and lumen 416 to be smaller in diameter than lumen 420 and/or lumen 424, the amount and/or size of lenticular material aspirated during phacoemulsification is limited or restricted at the distal end of the needle, tip 402, such that the proximal portions of the needle, shaft 404 and hub 406, do not receive a greater amount of lenticular material and/or lenticular material greater in size than can be handled by the length and diameter of lumen 420 and lumen 424. In some embodiments, the improved conductance of lenticular material during aspiration with phacoemulsification needle 400 overcomes the complications of existing phacoemulsification needle designs, including flare tip designs, in which the aspiration port and lumen at the distal end of the needle are greater in diameter than the proximal portions of the needle, the lumen of the shaft and/or the lumen of the hub, thereby making the needle more susceptible to clogging during aspiration.

In some embodiments, phacoemulsification needle 400 can prevent, or at least reduce, the need to increase the ultrasound energy and/or vacuum strength of a phacoemulsification machine for the purpose of dislodging a clog. This can prevent, or at least reduce, undesirable collateral damage to surrounding tissue including the iris and lens capsule.

In some embodiments, during phacoemulsification, once phacoemulsification needle 400 is inserted through the incision made in the capsular lens, beveled tip 414 can contour the convex lens to deliver ultrasound energy and facilitate sculpting and/or chopping.

In some embodiments, beveled tip 414 can assist in lens grooving and cracking during phacoemulsification. In some preferred embodiments, beveled tip 414 can improve grooving and cracking along branched lens sutures such as Y sutures.

In some embodiments, head 402 can assist in and improve lens grooving and cracking during phacoemulsification. In some preferred embodiments, head 402 can improve grooving and cracking along branched lens sutures such as Y sutures. In some embodiments, head 402 can prevent, or at least reduce, undesirable collateral damage to surrounding tissue including the iris and lens capsule during phacoemulsification.

In some embodiments, phacoemulsification needle 400 can decrease the amount of ultrasound energy (movement), aspiration rate, and/or vacuum needed during sculpting, chopping, and/or occlusion. This can prevent, or at least reduce the chance of, corneal incision contracture (corneal wound burn) resulting from ultrasound energy.

In some embodiments, phacoemulsification needle 400 is compatible with various phacoemulsification techniques including, but not limited to, grooving, sculpting, cracking, divide-and-conquer, stop-and-chop, occlusion, and/or carousel.

In some embodiments, phacoemulsification needle 400 can prevent, or at least reduce, the occurrence of iris tears, capsular tears, and/or capsular rupture and vitreous loss during phacoemulsification.

In some embodiments, phacoemulsification needle 400 can minimize, or at least reduce, the occurrence of postoperative vitreoretinal complications such as retinal detachment, cystoid macular edema, endophthalmitis, and/or endothelial cell loss.

In at least some embodiments, during phacoemulsification, as beveled tip 414 penetrates into the lens nucleus, occlusion is initiated. As head 402 advances into lenticular tissue, the expanded (the proximal end of the head is larger in diameter than the shaft of the needle) and/or tapered nature of the head creates a wedge into the tissue and applies force to the material in front of advancing beveled tip 414 to initiate and propagate cracking of the lens.

In some embodiments, the wedge effect of head 402 improves the efficacy of in longitudinal motion and/or rotational (Ozil) motion on lenticular tissue.

In some embodiments, the wedge effect of phacoemulsification needle 400 during occlusion improves the efficacy of chopping with a secondary instrument (such as a chopper).

In some embodiments, the wedge effect of phacoemulsification needle 400 can prevent, or reduce, the occurrence of coring. In some embodiments, phacoemulsification needle 400 can prevent, or at least reduce, the occurrence of post-occlusion surge of peripheral tissue such as the iris and/or capsule.

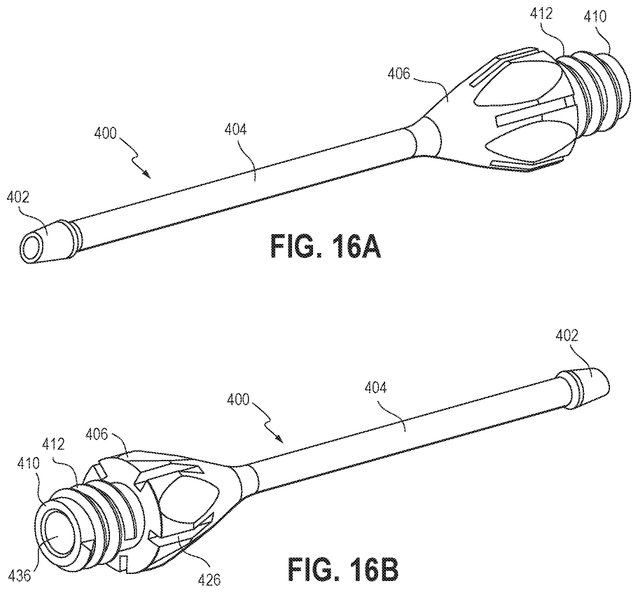

FIGS. 16A and 16B are perspective views of phacoemulsification needle 400. Opening 436 of lumen 424 can be seen in FIG. 16B.

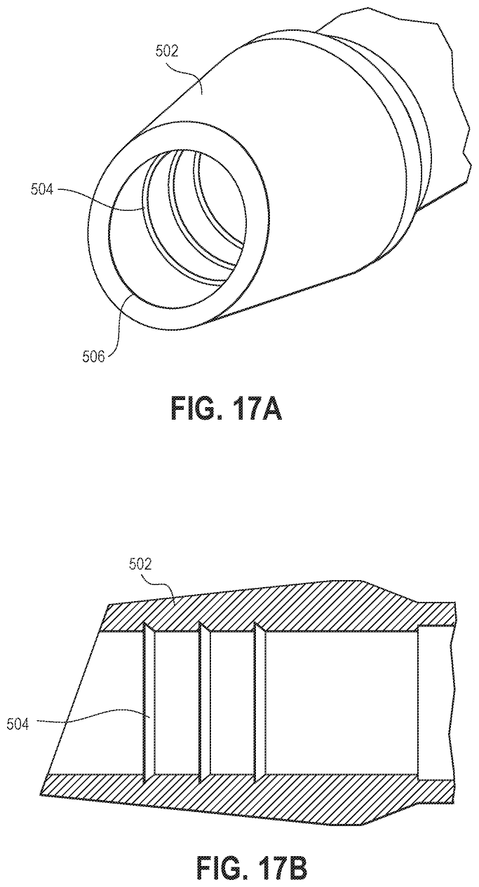

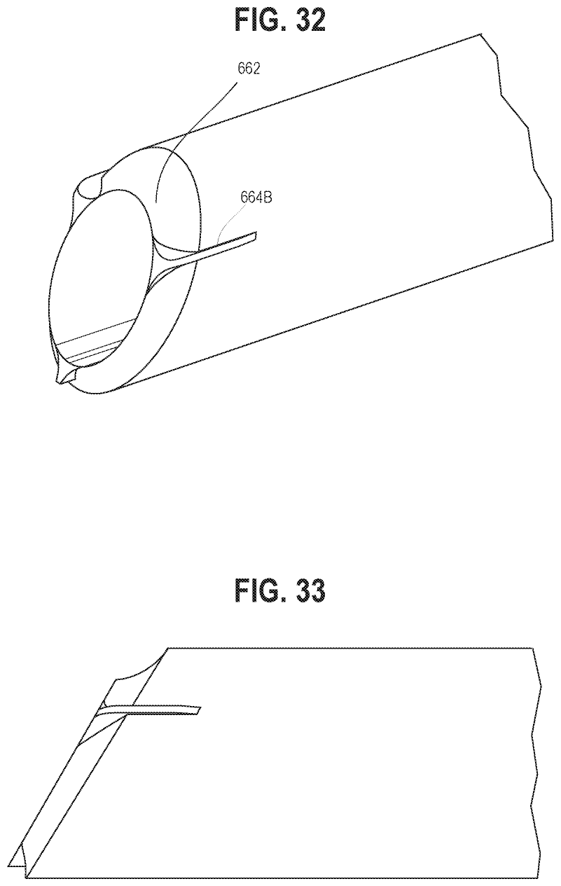

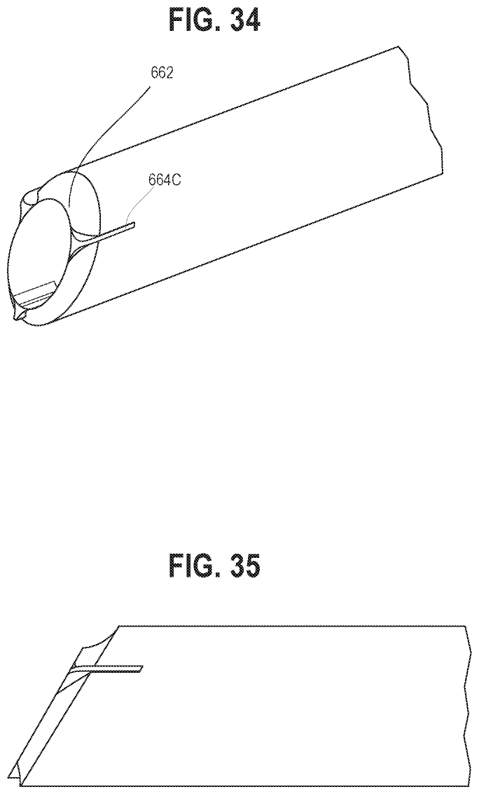

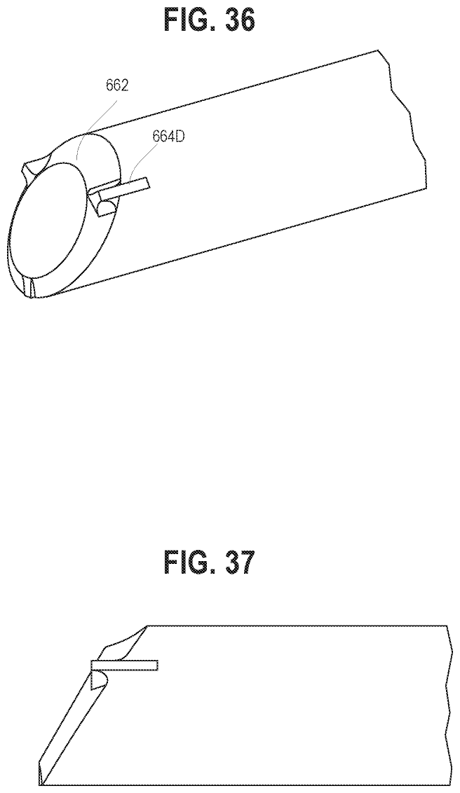

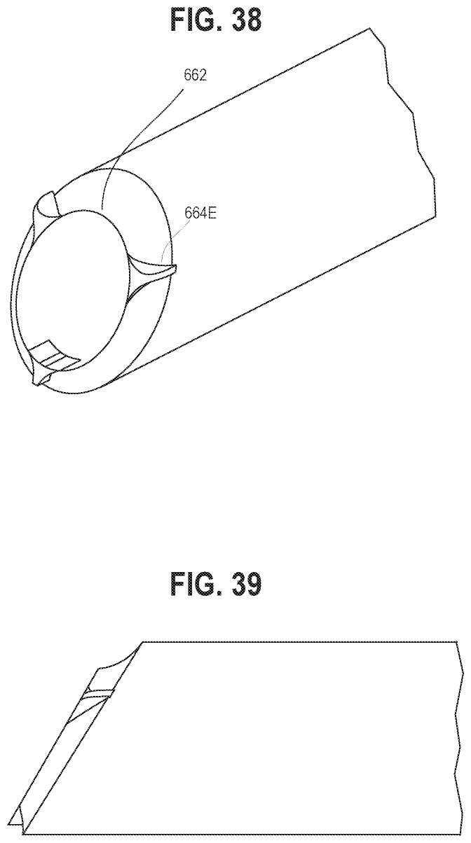

FIGS. 17A-25B and FIGS. 30-39 illustrate various features that can be implemented in a phacoemulsification needle head to assist in and improve lens grooving and cracking and/or engagement of target lenticular material during phacoemulsification. In some embodiments, these features can decrease the amount of ultrasound energy (movement), aspiration rate, and/or vacuum needed during sculpting, chopping, and/or occlusion. In some embodiments, these features can improve occlusion and/or followability during phacoemulsification.

In some embodiments, various combinations of these features can be implemented in the tapered and/or expanded (the proximal end of the head is larger in diameter than the shaft of the needle) head design of phacoemulsification needles disclosed herein. In some embodiments, these features, or combinations thereof, can improve longitudinal and rotational stability of the head for wedging.

In some embodiments, various combinations of these features can be implemented on the standard cylindrical geometry of existing phacoemulsification tips or needles.

FIGS. 17A and 17B illustrate head 502 that includes at least one internal, groove 504 situated around the circumference of lumen 506. In some embodiments, head 502 can be tapered and/or expanded (the proximal end of the head is larger in diameter than the shaft of the needle). In some embodiments, head 502 can promote engagement, occlusion, and followability of lenticular tissue during phacoemulsification.

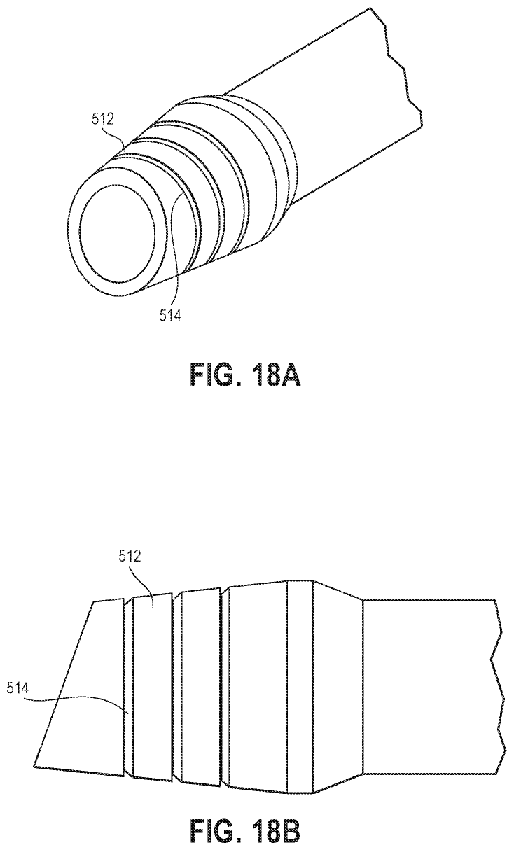

FIGS. 18A and 18B illustrate head 512 that includes at least one external groove 514 situated around the circumference of the head. In some embodiments, head 512 can be tapered and/or expanded. In some embodiments, head 512 can promote engagement, occlusion, and followability of lenticular tissue during phacoemulsification.

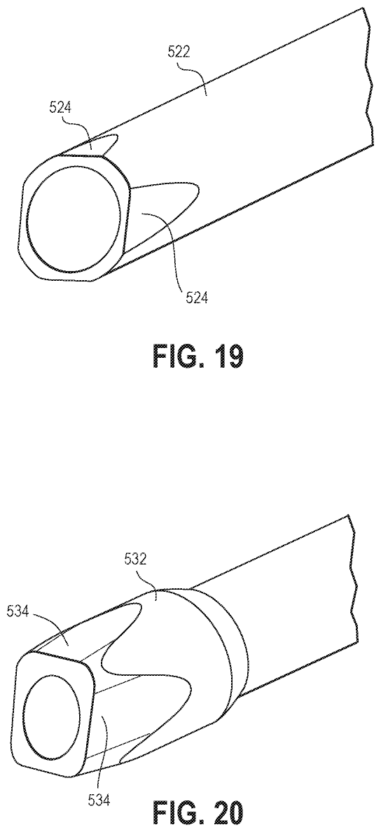

FIG. 19 illustrates head 522 that includes at least one external recess 524 situated at the distal end of head 522 and extending along at least a portion of the length of the needle. In some preferred embodiments, head 522 can include four recesses 524 spaced equidistant about the external surface of the head.

FIG. 20 illustrates head 532 that includes at least one external, longitudinal recess 534 extending at least a portion of the length of head 532. In some preferred embodiments, head 532 can include four recesses 534 spaced equidistant about the external surface of the head. In some embodiments, head 532 can be tapered and/or expanded. In some embodiments, the exterior of head 532 can be square in cross-section. In some embodiments, head 532 can improve lens cracking.

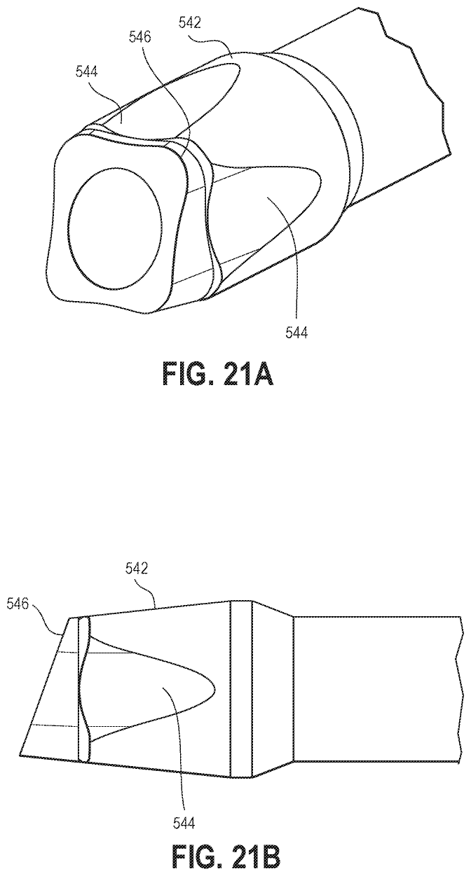

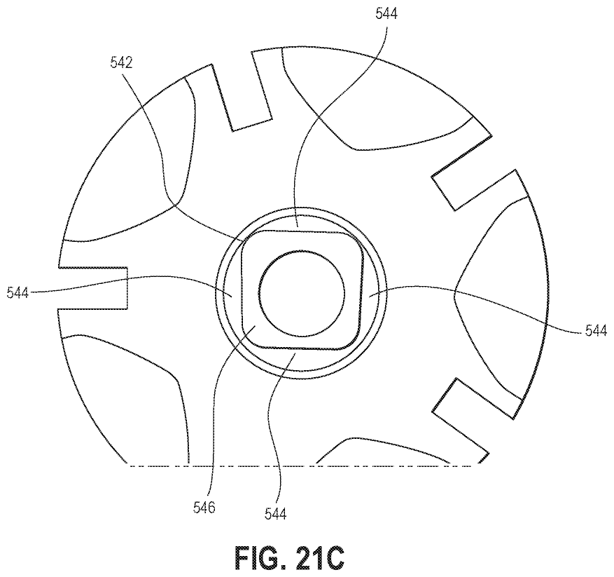

FIGS. 21A-C illustrate head 542 that includes at least one external, longitudinal recess 544 extending at least a portion of the length of head 542. In some preferred embodiments, head 542 can include four recesses 544 spaced equidistant about the external surface of the head. The distal end of head 542 can include chamfered edge 546. In some embodiments, head 542 can be tapered and/or expanded. In some embodiments, the exterior of head 542 can be square in cross-section. In some embodiments, head 542 can mediate wedging and cracking of lenticular tissue.

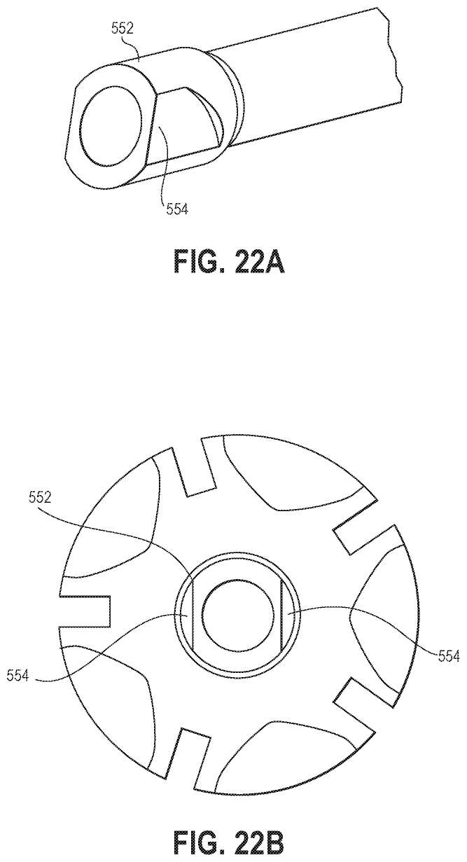

FIGS. 22A and 22B illustrate head 552 that includes at least one external, longitudinal recess 554 extending at least a portion of the length of the head. In some preferred embodiments, head 552 can include two recesses 554 spaced equidistant about the external surface of the head. In some embodiments, head 552 can be tapered and/or expanded. In some embodiments, recesses 554 prevent, or reduce, lenticular tissue from slipping up the sides of the tip. This can prevent, or reduce, the occurrence of coring.

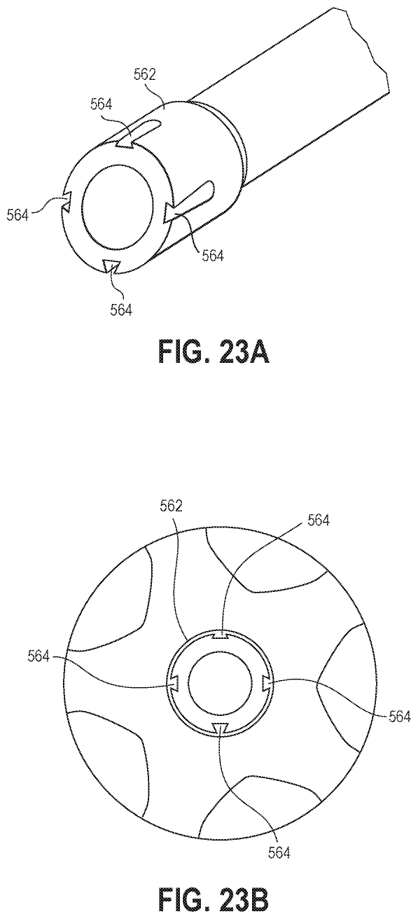

FIGS. 23A and 23B illustrate head 562 that includes at least one external, longitudinal groove 564 extending at least a portion of the length of head 562. In some preferred embodiments, head 562 can include four grooves 564 spaced equidistant about the external surface of the head. In some embodiments, head 562 can be tapered and/or expanded. In some applications, head 562 can improve occlusion. The shape of grooves 564 can be, but is not limited to, radiused, square, v-shaped, or triangular.

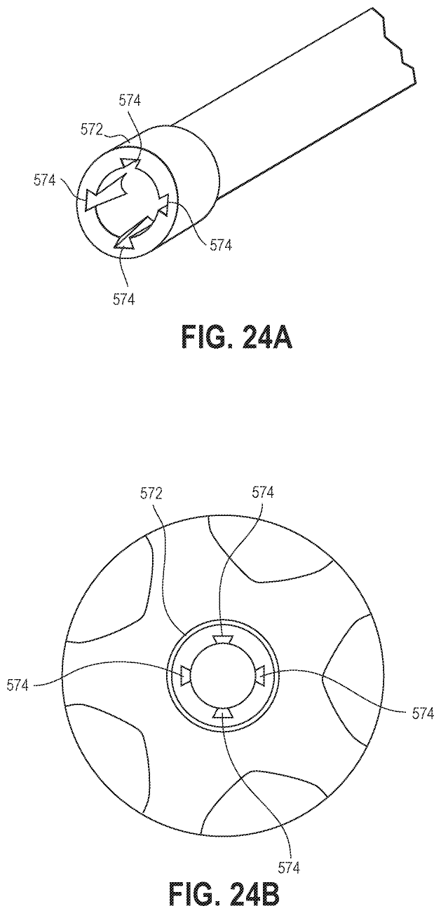

FIGS. 24A and 24B illustrate head 572 that includes at least one internal, longitudinal groove 574 extending at least a portion of the length of head 572. In some preferred embodiments, head 572 can include four grooves 574 spaced equidistant about the internal surface of the head. In some embodiments, head 572 can be tapered and/or expanded. In some applications, head 572 can improve occlusion and/or Ozil cutting. The shape of grooves 574 can be, but is not limited to, radiused, square, v-shaped, or triangular.

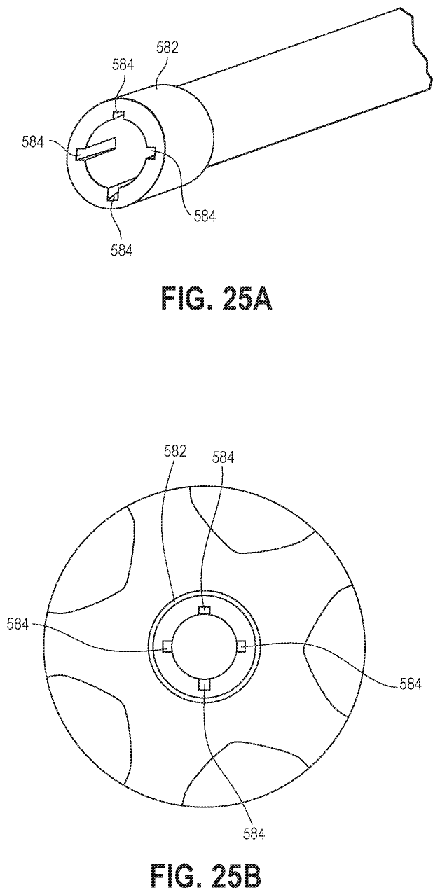

FIGS. 25A and 25B illustrate head 582 that includes at least one internal, longitudinal groove 584 extending at least a portion of the length of head 582. In some preferred embodiments, head 582 can include four grooves 584 spaced equidistant about the internal surface of the head. In some embodiments, head 582 can be tapered and/or expanded. The shape of grooves 584 can be, but is not limited to, radiused, square, v-shaped, or triangular.

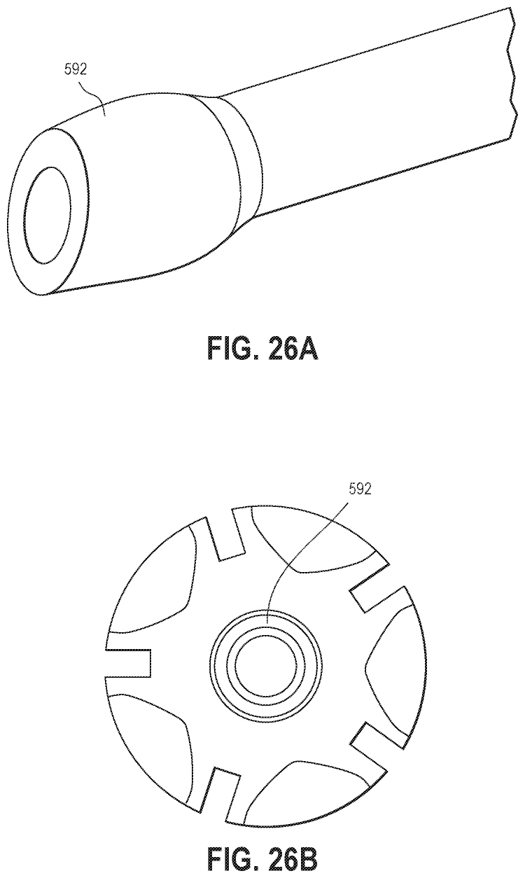

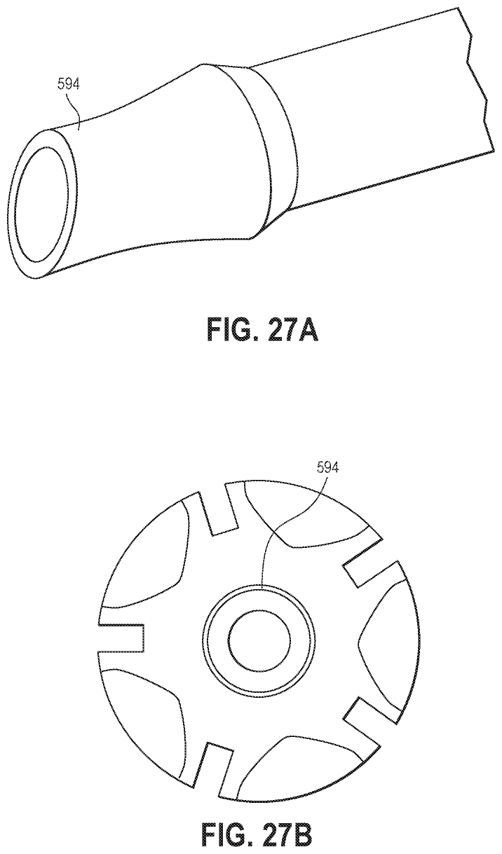

FIGS. 26A and 26B illustrate head 592 that has a convex shape. In some embodiments, head 592 can be tapered and/or expanded. FIGS. 27A and 27B illustrate head 594 that has a concave shape. In some embodiments, head 594 can be tapered and/or expanded. In some embodiments, the parabolic curvature of the phacoemulsification needle head can be adjusted to increase or decrease resistance to lenticular entry and/or removal during phacoemulsification.

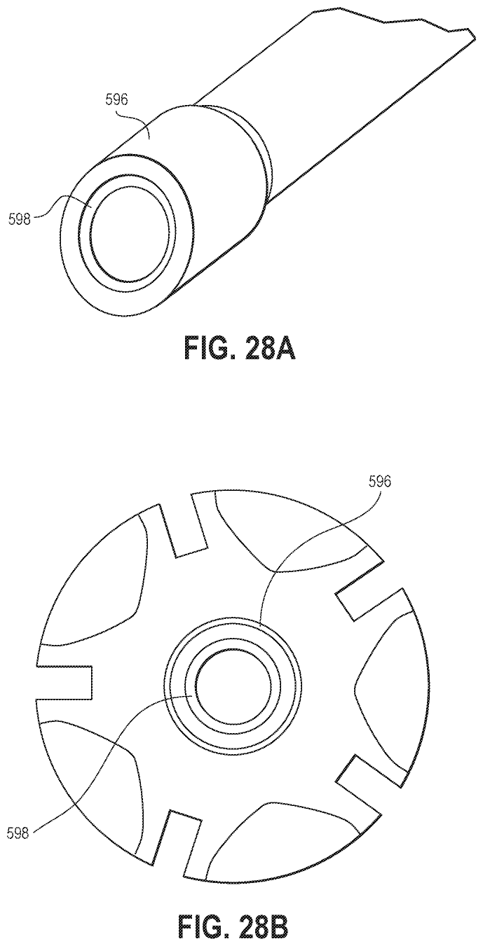

FIGS. 28A and 28B illustrate head 596 that includes internal chamfered edge 598 at the lumen opening of the aspiration port. In some embodiments, head 596 can be tapered and/or expanded. In some embodiments, chamfered edge 598 of the lumen opening can improve occlusion and followability by forming a temporary plug or seal between lenticular tissue and the lip of the tip while allowing aspiration of emulsified tissue. This can allow for a smoother passage of lenticular tissue into the lumen. In at least some embodiments, the tip of head 596 can be beveled.

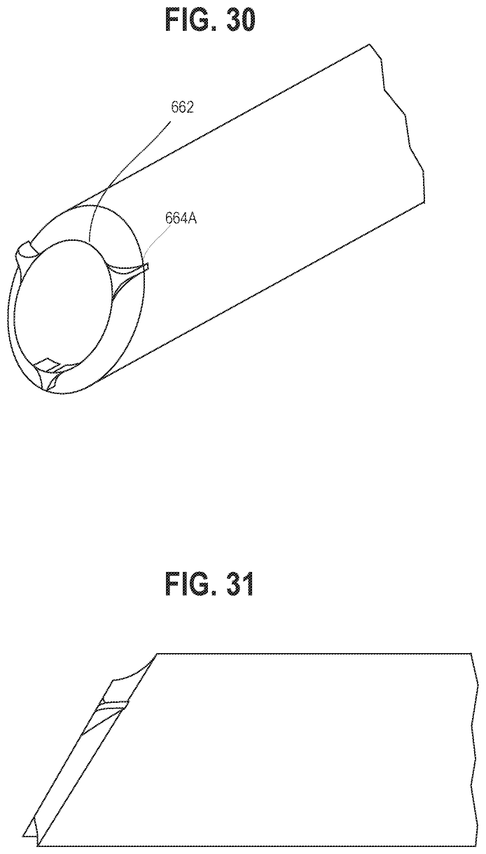

FIGS. 30 to 39 illustrate head 662 that includes at least one external, fin 664A-664E extending at least a portion of the length of head 662. In some embodiments, head 662 can include at least three fins spaced equidistant about the external surface of the head. In some embodiments, head 662 has exactly two, three, four, five, or six fins. In some embodiments, the fins are spaced equidistant about the external surface of the head. In some embodiments, the fins are not spaced equidistant about the external surface of the head. In some embodiments, head 662 can be tapered and/or expanded. In some applications, head 662 can improve occlusion.

The shape of fins 664A-664E can be, but is not limited to, radiused, square, v-shaped, curved, or triangular. In some embodiments, the fins on head 662 are symmetrical. In some embodiments, at least two of the fins on head 662 are different in shape.

In some embodiments, fins 664A-664E can stabilize tissue being vacuumed up during cataract surgery. In some embodiments, fins 664A-664E can stabilize tissue during quadrant removal. In at least some embodiments, fins 664A-664E keep tissue from rotating wildly and/or spinning and at the same time also help break the tissue apart. This fracturing can be used to removed the cataractous lens which needs to be cracked and pulled away from the delicate structures behind it. In some embodiments, fins 664A-664E guide the removed tissue.

In some embodiments, at least one of the fins can be combined with groove.

Various embodiments of a phacoemulsification needle, or portions thereof, including those described above, can be made from materials such as, but not limited to, metal, steel, and/or alloys. In some embodiments, the phacoemulsification needle can be made from titanium alloy comprising up to, and inclusive of, 6% aluminum by weight and up to, and inclusive of, 4% vanadium by weight.

While particular elements, embodiments and applications of the present invention have been shown and described, it will be understood, that the invention is not limited thereto since modifications can be made by those skilled in the art without departing from the scope of the present disclosure, particularly in light of the foregoing teachings.

What is claimed is:

1. A needle comprising:
(a) a head with a first lumen formed therein;
(b) a shaft with a second lumen formed therein, wherein said shaft is continuous with said head; and
(c) a hub with a third lumen formed therein, wherein said hub is continuous with said shaft;
wherein, the diameter of said third lumen is greater than the diameter of said first lumen and said second lumen and the diameter of said second lumen is greater than the diameter of said first lumen.

2. The needle of claim 1, wherein said head is tapered and comprises a proximal end and a distal end.

3. The needle of claim 2, wherein the circumference of said proximal end is greater than the circumference of said distal end.

4. The needle of claim 3, wherein the circumference of said proximal end of said head is greater then the circumference of said shaft.

5. The needle of claim 2, wherein said distal end comprises a beveled tip.

6. The needle of claim 5, wherein said beveled tip is at an angle of 20°.

7. The needle of claim 3, wherein said head further comprises a plurality of sides.

8. The needle of claim 7, wherein said plurality of sides comprises six sides and wherein the exterior surface of said head is hexagonal in cross-section.

9. The needle of claim 7, wherein said plurality of sides comprises four sides and wherein the exterior surface of said head is square in cross-section.

10. The needle of claim 7, wherein said plurality of sides comprises three sides and wherein the exterior surface of said head is triangular in cross-section.

11. The needle of claim 1, wherein said needle is attached to an ultrasonic phacoemulsification handpiece and used to emulsify and aspirate a cataract from an eye of a patient.

12. The needle of claim 11, wherein because the diameter of said third lumen is greater than the diameter of said first lumen and said second lumen and the diameter of said second lumen is greater than the diameter of said first lumen, said needle is prevented from clogging during aspiration of said cataract.

13. A phacoemulsification needle comprising:
(a) a head with a first lumen formed therein, said head comprising:
(i) a proximal end;
(ii) a distal end; and
(iii) a plurality of tapered sides;
(b) a shaft with a second lumen formed therein, wherein said shaft is continuous with said head; and
(c) a hub with a third lumen formed therein, wherein said hub is continuous with said shaft.

14. The phacoemulsification needle of claim 13, wherein the circumference of said proximal end of said head is greater than the circumference of said distal end of said head.

15. The phacoemulsification needle of claim 14, wherein the circumference of said proximal end of said head is greater than the circumference of said shaft.

16. The phacoemulsification needle of claim 13, wherein said head further comprises:
(iv) a beveled tip on said distal end.

17. The phacoemulsification needle of claim 14, wherein said plurality of tapered sides comprises six sides.

18. The phacoemulsification needle of claim 14, wherein said plurality of tapered sides comprises four sides.

19. The phacoemulsification needle of claim 14, wherein said phacoemulsification needle is attached to an ultrasonic phacoemulsification handpiece and used to emulsify and aspirate a cataract from an eye of a patient.

20. The phacoemulsification needle of claim 19, wherein said phacoemulsification needle can decrease an amount of ultrasonic energy, an aspiration rate, or a vacuum strength needed during sculpting, chopping, or occlusion of said cataract.
